(19) United States
(12) Patent Application Publication
(10) Pub. No.: US 2023/0357861 A1
(43) Pub. Date: Nov. 9, 2023

(54) NOTCH1 BIOMARKERS FOR CANCER THERAPY

(71) Applicant: Xiang LI, Beijing (CN)

(72) Inventor: Xiang LI, Beijing (CN)

(21) Appl. No.: 18/245,077

(22) PCT Filed: Sep. 16, 2021
(86) PCT No.: PCT/US21/50657

§ 371 Date: Mar. 13, 2023

(30) Foreign Application Priority Data
Sep. 23, 2020 (WO) PCT/CN2020/117167

(51) Int. Cl. C12Q 1/6886; G01N 33/50

(52) U.S. Cl. CPC C12Q 1/6886; G01N 33/5011; C12Q 2600/158; C12Q 2600/156; G01N 2500/10

(57) ABSTRACT

Provided is the use of NOTCH as a biomarker for the efficacy of YM155 monobromide in cancer therapy, and related kits, compositions, and methods for diagnosing and treating cancer in a subject in need thereof.

### CROSS-REFERENCE TO RELATED APPLICATIONS

This application claims priority to PCT/CN2020/117167, filed Sep. 23, 2020, which is incorporated by reference in its entirety.

### BACKGROUND

### Technical Field

Embodiments of the present disclosure relate to the use of NOTCH as a biomarker for the efficacy YM155 monobromide in cancer therapy, and related kits, compositions, and methods for diagnosing and treating cancer in a subject in need thereof.

### Description of the Related Art

YM155 monobromide is a small-molecule that exhibits potent antitumor activity (see, e.g., Minematsu et al., Drug Metabolism and Disposition, 37:619-628, 2008). YM-155 exerts anti-tumor effects in various in vivo cancer models, including prostate, pancreatic, and lung cancer (see, e.g., Nakahara et al., Cancer Research 67:8014-8021, 2007; and Na et al., PLoS One 7(6), 2012).

However, there is a need in the art to better predict the anti-cancer therapeutic efficacy of YM155 monobromide, and thereby identify patients that will benefit most from treatment with this chemotherapeutic, and others.

### BRIEF SUMMARY

Embodiments of the present disclosure include methods for treating a NOTCH-associated cancer in a subject in need thereof, comprising administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof, thereby treating the NOTCH-associated cancer in the subject in need thereof.

Some embodiments comprise the steps:
(a) determining (i) intracellular NOTCH (ICN) levels, (ii) NOTCH mutation status, and/or (iii) NOTCH gene copy number, in a sample of cancer tissue from the subject; and
(b) administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof, to the subject if (i) ICN levels in the cancer tissue are increased relative to a control or reference, (ii) if the cancer tissue comprises an activating NOTCH mutation relative to wild-type NOTCH, or (iii) if the NOTCH gene copy number is increased relative to that of a NOTCH copy gene reference.

Certain embodiments comprise administering to the subject a chemotherapeutic agent excluding (or other than) YM155 monobromide if (i) ICN levels in the cancer tissue are not substantially increased or undetectable relative to the control or reference, (ii) if the cancer tissue lacks an activating NOTCH mutation relative to wild-type NOTCH, and/or (iii) if NOTCH gene copy number is not substantially increased relative to that of the NOTCH copy gene reference.

Also included are methods for predicting therapeutic response to YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof, in a subject with cancer, comprising
(a) determining (i) intracellular NOTCH (ICN) levels, (ii) NOTCH mutation status, and/or (iii) NOTCH gene copy number, in a sample of cancer tissue from the subject; and
(b) (i) characterizing the subject as responsive to YM155 monobromide therapy if (i) ICN levels in the cancer tissue are increased relative to a control or reference, (ii) if the cancer tissue comprises an activating NOTCH mutation relative to wild-type NOTCH, or (iii) if the NOTCH gene copy number is increased relative to that of a NOTCH copy gene reference; or
   (ii) characterizing the subject as non-responsive to YM155 monobromide therapy if (i) ICN levels in the cancer tissue are not substantially increased or undetectable relative to the control or reference, (ii) if the cancer tissue lacks an activating NOTCH mutation relative to wild-type NOTCH, and/or (iii) if NOTCH gene copy number is not substantially increased relative to that of the NOTCH copy gene reference,
thereby predicting therapeutic response to YM155 monobromide in the subject with cancer. Some embodiments include administering YM155 monobromide to the subject if the subject is characterized as responsive to YM155 monobromide therapy. Certain embodiments include administering to the subject a chemotherapeutic agent excluding YM155 monobromide if the subject is characterized as non-responsive to YM155 monobromide therapy.

In some embodiments, the NOTCH is NOTCH1, and wherein the ICN levels are intracellular NOTCH1 (ICN1) levels. In some embodiments, the NOTCH is NOTCH3, and wherein the ICN levels are intracellular NOTCH3 (ICN3) levels.

Some embodiments comprise determining ICN levels, optionally ICN1 or ICN3 levels, in sample of cancer tissue by immunohistochemistry (IHC), for example, chromogenic or fluorescent IHC, enzyme linked immunosorbent assay (ELISA), or Western blot on a human NOTCH protein or gene. Some embodiments comprise administering YM155 to the subject if ICN levels, including ICN1 or ICN3 levels, in the cancer tissue are increased by about or at least about 1.2, 1.3, 1.4, 1.5, 2, 3, 4, 5, 6, 7, 8, 9, or 10, 50, or 100-fold or more relative to the ICN levels of the control or reference, optionally wherein the control is a healthy tissue.

Some embodiments comprise determining NOTCH mutation status, including NOTCH1 or NOTCH3 mutation status, in the sample of cancer tissue in situ hybridization (ISH), fluorescence in situ hybridization (FISH), whole exome sequencing (WES), single nucleotide polymorphism (SNP) array, next generation sequencing (NGS), or comparative genome hybridization (CGH) on a human NOTCH protein or gene. In certain embodiments, the NOTCH is NOTCH1, and the activating NOTCH1 mutation is a ligand-independent activation (LIA) NOTCH1 mutant or an impaired degradation (ID) of NOTCH1 mutant. In certain embodiments, the activating NOTCH1 mutation is selected from one or more of one or more of a heterodimerization domain (HD) mutant, optionally a single nucleotide polymorphism (SNP) in the HD or a small in-frame insertion or deletion in the HD, a juxtamembrane expansion (JME) allele, and a C-terminal Pro-Glu-Ser-Thr-rich (PEST) domain mutant. In certain embodiments, the NOTCH is NOTCH3, and the activating NOTCH3 mutation is a C-terminal Pro-Glu-Ser-Thr-rich (PEST) domain mutant.

Some embodiments comprise determining NOTCH gene copy number, optionally NOTCH1 or NOTCH3 gene copy number, in the sample of cancer tissue by array comparative genome hybridization (aCGH), single nucleotide polymorphism (SNP) array, copy number variation (CNV) sequencing, or multiplex ligation-dependent probe amplification (MLPA). In certain embodiments, the NOTCH gene copy number, for example, the NOTCH1 or NOTCH3 gene copy number, in the sample of cancer tissue is increased by about or at least about 1.5, 2, 3, 4, 5, 6, 7, 8, 9, or 10-fold relative to that of the NOTCH gene copy number reference. Some embodiments comprise obtaining the NOTCH gene copy number reference, including the NOTCH1 or NOTCH3 gene copy number reference, from a database, or determining the NOTCH gene copy number reference from a non-cancerous tissue from a control, optionally by aCGH, SNP array, CNV sequence, or MLPA.

Some embodiments include obtaining the sample of cancer tissue from the subject. In certain embodiments, the sample of cancer tissue is a blood sample, a surgical sample, a biopsy sample, a pleural effusion sample, or an ascetic fluid sample obtained from the subject, optionally selected from one or more of a white blood cell, breast, lung, gastrointestinal (stomach, colon, rectal), ovarian, pancreatic, liver, bladder, cervical, neuronal, uterine, salivary gland, kidney, prostate, thyroid, or muscle tissue sample. In certain embodiments, the subject is a human subject.

In certain embodiments, the cancer is selected from one or more of a leukemia, lymphoma (including Hodgkin's lymphoma and Non-Hodgkin's lymphoma), carcinoma, sarcoma such as rhabdomyosarcoma for example, alveolar rhabdomyosarcoma, (including sarcoma originating in the bones, tendons, cartilage, muscle, fat, fibrous, blood vessels, adipose, and/or connective tissue), breast cancer (including metastatic breast cancer), lung cancer (including non-small cell lung cancer, small cell lung cancer, adenocarcinoma, and squamous carcinoma of the lung), neuroblastoma, medulloblastoma, astrocytoma, glioblastoma multiforme, retinoblastoma, myeloma, adenosquamous carcinoma, carcinosarcoma, mixed mesodermal tumor, teratocarcinoma), gastrointestinal cancer, stomach cancer, colorectal cancer, colon cancer, rectal cancer, ovarian cancer, pancreatic cancer, liver cancer, bladder cancer, cervical cancer, glioblastoma, uterine carcinoma, salivary gland carcinoma, cancer. In specific embodiments, the cancer is a leukemia or lymphoma, optionally selected from T-cell lymphoblastic leukemia (T-ALL), T-cell lymphoblastic lymphoma (T-LBL), and chronic lymphocytic leukemia (CLL), a non small cell lung cancer, or a breast cancer. In certain embodiments, the T-ALL is a NOTCH1-associated T-ALL, or a NOTCH3-associated T-ALL. In certain embodiments, the breast cancer is NOTCH-1 associated breast cancer, or a NOTCH3-associated breast cancer, optionally triple-negative breast cancer.

Particular embodiments relate to the use of a diagnostic kit for determining therapeutic response to YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof, therapy in a subject with cancer, comprising means for determining (i) intracellular NOTCH (ICN) levels, (ii) NOTCH mutation status, and/or (iii) NOTCH gene copy number, in a sample of cancer tissue from the subject, including cancer tissue and non-cancerous tissue. In certain embodiments, the NOTCH is NOTCH1. In some embodiments, the NOTCH is NOTCH3.

In certain embodiments, the means for determining ICN levels comprise reagents for performing a diagnostic assay selected from one or more of immunohistochemistry (IHC) optionally chromogenic or fluorescent IHC, enzyme linked immunosorbent assay (ELISA), or Western blot on a human NOTCH protein. In certain embodiments, the means for determining NOTCH mutation status comprise reagents for performing a diagnostic assay selected from one or more of in situ hybridization (ISH), fluorescence in situ hybridization (FISH), whole exome sequencing (WES), single nucleotide polymorphism (SNP) array, next generation sequencing (NGS), or comparative genome hybridization (CGH) on a human NOTCH protein or gene. In certain embodiments, the means for determining NOTCH gene copy number comprise reagents for performing a diagnostic assay selected from one or more of array comparative genome hybridization (aCGH), single nucleotide polymorphism (SNP) array, copy number variation (CNV) sequencing, or multiplex ligation-dependent probe amplification (MLPA).

In certain embodiments, the cancer is selected from one or more of a leukemia, lymphoma (including Hodgkin's lymphoma and Non-Hodgkin's lymphoma), carcinoma, sarcoma such as rhabdomyosarcoma for example, alveolar rhabdomyosarcoma, (including sarcoma originating in the bones, tendons, cartilage, muscle, fat, fibrous, blood vessels, adipose, and/or connective tissue), breast cancer (including metastatic breast cancer), lung cancer (including non-small cell lung cancer, small cell lung cancer, adenocarcinoma, and squamous carcinoma of the lung), neuroblastoma, medulloblastoma, astrocytoma, glioblastoma multiforme, retinoblastoma, myeloma, adenosquamous carcinoma, carcinosarcoma, mixed mesodermal tumor, teratocarcinoma), gastrointestinal cancer, stomach cancer, colorectal cancer, colon cancer, rectal cancer, ovarian cancer, pancreatic cancer, liver cancer, bladder cancer, cervical cancer, glioblastoma, uterine carcinoma, salivary gland carcinoma, cancer. In certain embodiments, the cancer is a leukemia or lymphoma, optionally selected from T-cell lymphoblastic leukemia (T-ALL), T-cell lymphoblastic lymphoma (T-LBL), chronic lymphocytic leukemia (CLL), a non small cell lung cancer, or a breast cancer. In specific embodiments, the T-ALL is a NOTCH1-associated T-ALL, or a NOTCH3-associated T-ALL. In some embodiments, the breast cancer is NOTCH-1 associated breast cancer, or a NOTCH3-associated breast cancer, optionally triple-negative breast cancer.

Also included are patient care kits, comprising:
(a) means for determining (i) intracellular NOTCH (ICN) levels, (ii) NOTCH mutation status, and/or (iii) NOTCH gene copy number, in a sample of tissue from a subject, including cancer tissue and non-cancerous tissue; and
(b) YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof.

In certain embodiments, the NOTCH is NOTCH1. In certain embodiments, the NOTCH is NOTCH3. In certain embodiments, the means for determining ICN levels comprise reagents for performing a diagnostic assay selected from one or more of immunohistochemistry (IHC) optionally chromogenic or fluorescent IHC, enzyme linked immunosorbent assay (ELISA), or Western blot on a human NOTCH protein. In certain embodiments, the means for determining NOTCH mutation status comprise reagents for performing a diagnostic assay selected from one or more of in situ hybridization (ISH), fluorescence in situ hybridization (FISH), whole exome sequencing (WES), single nucleotide polymorphism (SNP) array, next generation sequencing (NGS), or comparative genome hybridization (CGH) on a human NOTCH protein or gene. In certain embodiments, the means for determining NOTCH gene copy number comprise reagents for performing a diagnostic assay selected from one or more of array comparative genome hybridization (aCGH), single nucleotide polymorphism (SNP) array, copy number variation (CNV) sequencing, or multiplex ligation-dependent probe amplification (MLPA).

Some embodiments include a pharmaceutical composition for use in treating a NOTCH-associated cancer, comprising YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof. In certain embodiments, the NOTCH-associated cancer is a NOTCH1-associated cancer, and/or a NOTCH3-associated cancer, optionally a T-ALL or a breast cancer.

Certain embodiments include the use of a composition in the preparation of a medicament for treating a NOTCH-associated cancer, comprising YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof. In specific embodiments, the NOTCH-associated cancer is a NOTCH1-associated cancer, and/or a NOTCH3-associated cancer, optionally a T-ALL or a breast cancer.

### BRIEF DESCRIPTION OF THE DRAWINGS

FIG. 1A shows the chemical structure of YM155 monobromide (CAS 781661-94-7).

FIG. 1B provides genomic information for the human NOTCH1 gene, and FIG. 1C provides genomic information for the human NOTCH3 gene.

FIG. 2 shows that YM155 inhibited cell proliferation of human T-lineage acute lymphoblastic leukemia (T-ALL) cells. CCRF-CEM, Jurkat, MOLT-4, I2.1 (CRL-2572), and LOUCY cells were cultured in 96-well plates and treated with YM155 at indicated doses (nM). Cell proliferation was detected by XTT assay.

FIG. 3 shows a Western blot analysis of intracellular cleaved and activated NOTCH1 (ICN1) in T-ALL cell lines, specifically CCRF-CEM, Jurkat, MOLT-4, I2.1 (CRL-2572), and LOUCY cells. GAPDH is shown as loading control.

FIGS. 4A-4D show that YM155 strongly-induced apoptosis of T-ALL cells that are positive for ICN1. By comparison, FIG. 4E shows that YM155 induced apoptosis at much lower levels in T-ALL cells that are negative for ICN1. Cells were treated with YM155 as indicated concentration for 72 hours. Apoptosis was quantified by Annexin V/PI staining and analyzed by flow cytometry.

FIG. 5 is a Western blot analysis that shows reduced ICN1 levels after 72 hour treatment with YM155 (10 nM) in T-ALL cell lines. GAPDH is shown as loading control.

FIG. 6 shows a Western blot analysis of intracellular cleaved and activated NOTCH1 (ICN1) and NOTCH3 (ICN3) in breast cancer cell lines, specifically AU656, T47D, HCC1143, and MDA-MB-157.

FIGS. 7A-7B show results of an apoptosis assay with HCC1143 cells using mitomycin C as a positive control; 7A shows the Caspase 3 activity assay, and 7B shows the Annexin V apoptosis assay.

FIG. 8 shows the analysis of apoptosis in YM155-treated breast cancer cells by Annexin V and Caspase 3 assays.

### DETAILED DESCRIPTION

Embodiments of the present disclosure relate to the surprising discovery that aberrant NOTCH signaling in human malignancies associates with increased anti-cancer efficacy of YM155 monobromide, and can therefore be used as a companion diagnostic or biomarker to optimize YM155-related cancer therapies.

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

For the purposes of the present disclosure, the following terms are defined below.

The articles “a” and “an” are used herein to refer to one or to more than one (i.e., to at least one) of the grammatical object of the article. By way of example, “an element” means one element or more than one element.

An “antagonist” or “inhibitor” refers to biological structure or chemical agent that interferes with or otherwise reduces the physiological action of another molecule, such as a protein. In some instances, the antagonist or inhibitor specifically binds to the other molecule and/or a functional ligand of the other molecule. In some instances, the antagonist or inhibitor down-regulates the expression of the other molecule. Included are full and partial antagonists.

An “agonist” or “activator” refers to biological structure or chemical agent that increases or enhances the physiological action of another agent or molecule. In some instances, the agonist specifically binds to the other agent or molecule. Included are full and partial agonists.

By “about” is meant a quantity, level, value, number, frequency, percentage, dimension, size, amount, weight or length that varies by as much as 20, 15, 10, 9, 8, 7, 6, 5, 4, 3, 2, or 1% to a reference quantity, level, value, number, frequency, percentage, dimension, size, amount, weight, or length.

The term “binding” refers to a direct association between two molecules, due to, for example, covalent, electrostatic, hydrophobic, and ionic and/or hydrogen-bond interactions, including interactions such as salt bridges and water bridges.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “half maximal effective concentration” or “EC₅₀” refers to the concentration of an agent as described herein at which it induces a response halfway between the baseline and maximum after some specified exposure time; the EC₅₀ of a graded dose response curve therefore represents the concentration of a compound at which 50% of its maximal effect is observed. EC₅₀ also represents the plasma concentration required for obtaining 50% of a maximum effect in vivo. Similarly, the “EC₉₀” refers to the concentration of an agent or composition at which 90% of its maximal effect is observed. The “EC₉₀” can be calculated from the “EC₅₀” and the Hill slope, or it can be determined from the data directly, using routine knowledge in the art. In some embodiments, the EC₅₀ of an agent is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, an agent will have an EC₅₀ value of about 1 nM or less.

The “half maximal inhibitory concentration” (or “IC₅₀”) is a measure of the potency of an agent in inhibiting a specific biological or biochemical function. This quantitative measure indicates how much of a particular agent (inhibitor) is needed to inhibit a given biological process (or component of a process, i.e. an enzyme, cell, cell receptor or microorganism) by half. The values are typically expressed as molar concentration. The concentration is commonly used as a measure of antagonist drug potency in pharmacological research. In some instances, IC₅₀ represents the concentration of an agent that is required for 50% inhibition in vitro. The IC₅₀ of an agent can be determined by constructing a dose-response curve and examining the effect of different concentrations of the agent on the desired activity, for example, inhibition of tumor cell proliferation, tumor-cell killing.

The “half-life” of an agent refers to the time it takes for the agent to lose half of its pharmacologic, physiologic, or other activity, relative to such activity at the time of administration into the serum or tissue of an organism, or relative to any other defined time-point. “Half-life” can also refer to the time it takes for the amount or concentration of an agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an amount that is about or at least about 1.1, 1.2, 1.5, 2, 3, 4, 5, 6, 7, 8, 9, 10, 15, 20, 30, 40, 50, 60, 70, 80, 90, 100, 200, 300, 400, 500, 600, 700, 800, 900, 1000, 2000, 3000, 4000, 5000-fold or more of the amount produced by no composition or a control composition (e.g., the absence of agent or a different agent). An “increased,” “stimulated” or “enhanced” amount may also include an amount that is about or at least about 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, 2000%, 3000%, 4000%, 5000% or more of the amount produced by no composition or a control composition. A “decreased” or “reduced” amount is typically a “statistically significant” amount, and may include an amount that is about or at least about 1.1, 1.2, 1.5, 2, 3, 4, 5, 6, 7, 8, 9, 10, 15, 20, 30, 40, 50, 60, 70, 80, 90, 100, 200, 300, 400, 500, 600, 700, 800, 900, 1000, 2000, 3000, 4000, or 5000-fold less of the amount produced by no composition or a control composition. A “decreased” or “reduced” amount may also include a 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%, 200%, 300%, 400%, 500%, 600%, 700%, 800%, 900%, 1000%, 2000%, 3000%, 4000%, or 5000% less of the amount produced by no composition or a control composition. Examples of comparisons and “statistically significant” amounts are described herein.

“Prodrug” is meant to indicate a compound that may be converted under physiological conditions or by solvolysis to a biologically active compound described herein, for example, a GSI compound. Thus, the term “prodrug” refers to a metabolic precursor of a compound that is pharmaceutically acceptable. A prodrug may be inactive when administered to a subject in need thereof, but is converted in vivo to an active compound. Prodrugs may be rapidly transformed in vivo to yield the parent compound, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987. Examples of prodrugs include, but are not limited to, acetate, formate, and benzoate derivatives of alcohol or amide derivatives of amine functional groups in the compounds of the disclosure and the like.

The term “prodrug” is also meant to include any covalently bonded carriers, which release the active compound in vivo when such prodrug is administered to a subject. Prodrugs of a compound may be prepared by modifying functional groups present in the compound in such a way that the modifications are cleaved, either in routine manipulation or in vivo, to the parent compound. Prodrugs include compounds where a hydroxy, amino, or mercapto group is bonded to any group that, when the prodrug of the compound is administered to a subject, cleaves to form a free hydroxy, free amino, or free mercapto group, respectively.

“Pharmaceutically-acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier, for example, which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

As used herein, the term “solvate” refers to an aggregate that comprises one or more molecules of a compound described herein with one or more molecules of solvent. The solvent may be water, in which case the solvate may be a hydrate. Alternatively, the solvent may be a biologically-inert organic solvent. Thus, the compounds described herein may exist as a hydrate, including a monohydrate, dihydrate, hemihydrate, sesquihydrate, trihydrate, tetrahydrate and the like, as well as the corresponding solvated forms. The compound of the disclosure may be true solvates, while in other cases, the compound may merely retain adventitious water or be a mixture of water plus some adventitious solvent.

A “pharmaceutical composition” refers to a formulation of a YM155 compound described herein and a medium generally accepted in the art for the delivery of the biologically active compound to mammals, e.g., humans. Such a medium includes all pharmaceutically acceptable carriers, diluents, and excipients.

The YM155 compounds described herein, or their pharmaceutically-acceptable salts, may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

In certain embodiments, the “purity” of any given agent in a composition may be defined. For instance, certain compositions may comprise an agent that is at least 80%, 85%, 90%, 91%, 92%, 93%, 94%, 95%, 96%, 97%, 98%, 99%, or 100% pure on a weight-weight basis, including all decimals and ranges in between, as measured, for example and by no means limiting, by high performance liquid chromatography (HPLC), a well-known form of column chromatography used frequently in biochemistry and analytical chemistry to separate, identify, and quantify compounds.

The term “solubility” refers to the property of an agent provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCl (with or without NaPO4). In specific embodiments, solubility is measured at relatively lower pH (e.g., pH 6.0) and relatively higher salt (e.g., 500 mM NaCl and 10 mM NaPO4). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37° C.

“Stable compound” and “stable structure” are meant to indicate a compound that is sufficiently robust to survive isolation to a useful degree of purity from a reaction mixture, and formulation into a therapeutic agent.

The term “polynucleotide” and “nucleic acid” includes mRNA, RNA, cRNA, cDNA, and DNA including genomic DNA. The term typically refers to polymeric form of nucleotides of at least 10 bases in length, either ribonucleotides or deoxynucleotides or a modified form of either type of nucleotide. The term includes single and double stranded forms of DNA.

A “gene” refers to a hereditary unit consisting of a sequence of DNA that occupies a specific location on a chromosome and codes for a functional molecule or protein. The structure of a gene consists of many elements of which the actual protein coding sequence is often only a small part. These elements include DNA regions that are not transcribed as well as untranslated regions of the RNA. Additionally, genes can have expression-altering regulatory regions that lie many kilobases upstream or downstream of the coding sequence. The information in a gene can also be represented by (or found in) a sequence of RNA or encoded protein.

A “subject” or a “subject in need thereof” includes a mammalian subject such as a human subject.

By “statistically significant” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95%, 96%, 97%, 98%, 99% or greater of some given reference quantity, level, value, number, frequency, percentage, dimension, size, amount, weight, length, or other.

A “stereoisomer” refers to a compound made up of the same atoms bonded by the same bonds but having different three-dimensional structures, which are not interchangeable. The present disclosure includes various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present disclosure includes tautomers of any said compounds.

“Therapeutic response” refers to improvement of symptoms (whether or not sustained) based on the administration of the therapeutic response.

As used herein, the terms “therapeutically effective amount”, “therapeutic dose,” “prophylactically effective amount,” or “diagnostically effective amount” is the amount of an agent needed to elicit the desired biological response following administration.

As used herein, “treatment” of a subject (e.g. a mammal, such as a human) or a cell is any type of intervention used in an attempt to alter the natural course of the subject or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied to every other embodiment unless expressly stated otherwise.

Certain embodiments include methods for treating a NOTCH-associated cancer in a subject in need thereof, comprising administering YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof, thereby treating the NOTCH-associated cancer in the subject in need thereof. The term “NOTCH-associated cancer” includes a cancer in which (i) intracellular NOTCH (ICN) levels in the cancer tissue are increased relative to a control or reference, (ii) the cancer tissue comprises an activating NOTCH mutation relative to wild-type NOTCH, and/or (iii) the NOTCH gene copy number is increased relative to that of a NOTCH copy gene reference.

Some methods of treating a NOTCH-associated cancer include the steps:
(a) determining (i) ICN levels, (ii) NOTCH mutation status, and/or (iii) NOTCH gene copy number, in a sample of cancer tissue from the subject; and
(b) administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof, to the subject if (i) ICN levels in the cancer tissue are increased relative to a control or reference, (ii) if the cancer tissue comprises an activating NOTCH mutation relative to wild-type NOTCH, or (iii) if the NOTCH gene copy number is increased relative to that of a NOTCH copy gene reference. Some embodiments include administering to the subject a chemotherapeutic agent excluding (or other than) YM155 monobromide if (i) ICN levels in the cancer tissue are not substantially increased or undetectable relative to the control or reference, (ii) if the cancer tissue lacks an activating NOTCH mutation relative to wild-type NOTCH, and/or (iii) if NOTCH gene copy number is not substantially increased relative to that of the NOTCH copy gene reference.

Also included are methods for predicting therapeutic response to YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof, in a subject with cancer, comprising:
(a) determining (i) ICN levels, (ii) NOTCH mutation status, and/or (iii) NOTCH gene copy number, in a sample of cancer tissue from the subject; and
(b) (i) characterizing the subject as responsive to YM155 monobromide therapy if (i) ICN levels in the cancer tissue are increased relative to a control or reference, (ii) if the cancer tissue comprises an activating NOTCH mutation relative to wild-type NOTCH, or (iii) if the NOTCH gene copy number is increased relative to that of a NOTCH copy gene reference; or
   (ii) characterizing the subject as non-responsive to YM155 monobromide therapy if (i) ICN levels in the cancer tissue are not substantially increased or undetectable relative to the control or reference, (ii) if the cancer tissue lacks an activating NOTCH mutation relative to wild-type NOTCH, and/or (iii) if NOTCH gene copy number is not substantially increased relative to that of the NOTCH copy gene reference,
thereby predicting therapeutic response to YM155 monobromide in the subject with cancer. Some embodiments include administering YM155 monobromide to the subject if the subject is characterized as responsive to YM155 monobromide therapy. Some instances include administering to the subject a chemotherapeutic agent excluding YM155 monobromide if the subject is characterized as non-responsive to YM155 monobromide therapy.

The Notch signaling pathway is a highly conserved cell signaling system. For example, mammals have four different notch receptors, referred to as NOTCH1, NOTCH2, NOTCH3, and NOTCH4. The notch receptor is a single-pass transmembrane receptor protein; including a hetero-oligomer composed of a large extracellular portion, which associates in a calcium-dependent, non-covalent interaction with a smaller piece of the notch protein composed of a short extracellular region, a single transmembrane-pass, and a small intracellular region.

Certain embodiments include treating a “NOTCH1-associated cancer”. Notch homolog 1, translocation-associated (Drosophila), or “NOTCH1”, is a human gene encoding a single-pass transmembrane receptor (see UniProtKB-P46531). NOTCH1 is Type 1 transmembrane protein that comprises an extracellular domain (ECD) consisting of multiple epidermal growth factor-like (EGF) repeats, and an intracellular domain (ICD) consisting of multiple, different domain types. NOTCH1 protein is synthesized in the endoplasmic reticulum as an inactive form which is proteolytically cleaved by a furin-like convertase in the trans-Golgi network before it reaches the plasma membrane to yield an active, ligand-accessible form. Cleavage results in a C-terminal fragment N (TM) and a N-terminal fragment N (EC). Following ligand binding, it is cleaved by ADAM17 to yield a membrane-associated intermediate fragment called notch extracellular truncation (NEXT) (PubMed:24226769). Following endocytosis, this fragment is then cleaved by one of the catalytic subunits of gamma-secretase (PSEN1 or PSEN2), to release a Notch-derived peptide containing the intracellular domain (ICD1, or NICD1) from the membrane. NOTCH1 functions as a receptor for membrane-bound ligands Jagged-1 (JAG1), Jagged-2 (JAG2), and Delta-1 (DLL1) to regulate cell-fate determination. Upon ligand activation through the released NICD1) it forms a transcriptional activator complex with RBPJ/RBPSUH and activates genes of the enhancer of split locus. NOTCH1 modulates the implementation of differentiation, proliferation, and apoptotic programs.

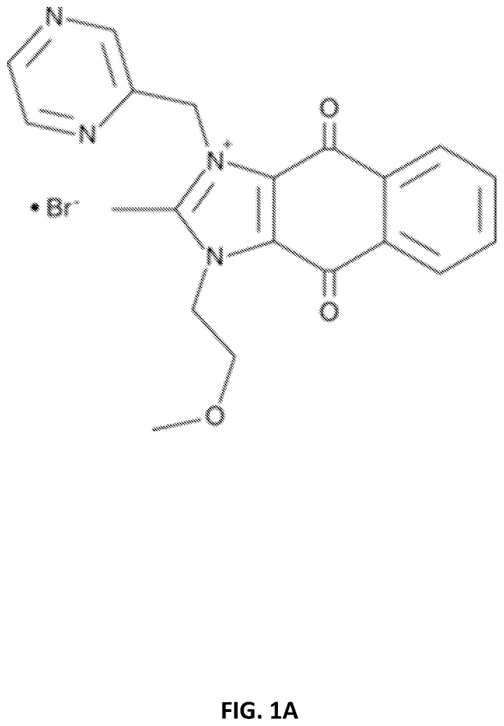

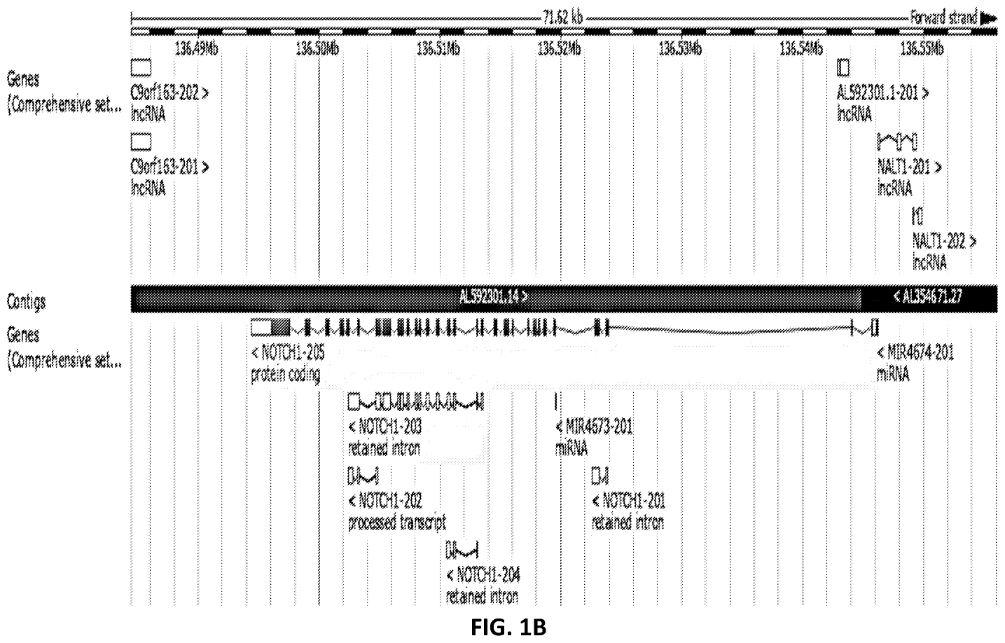

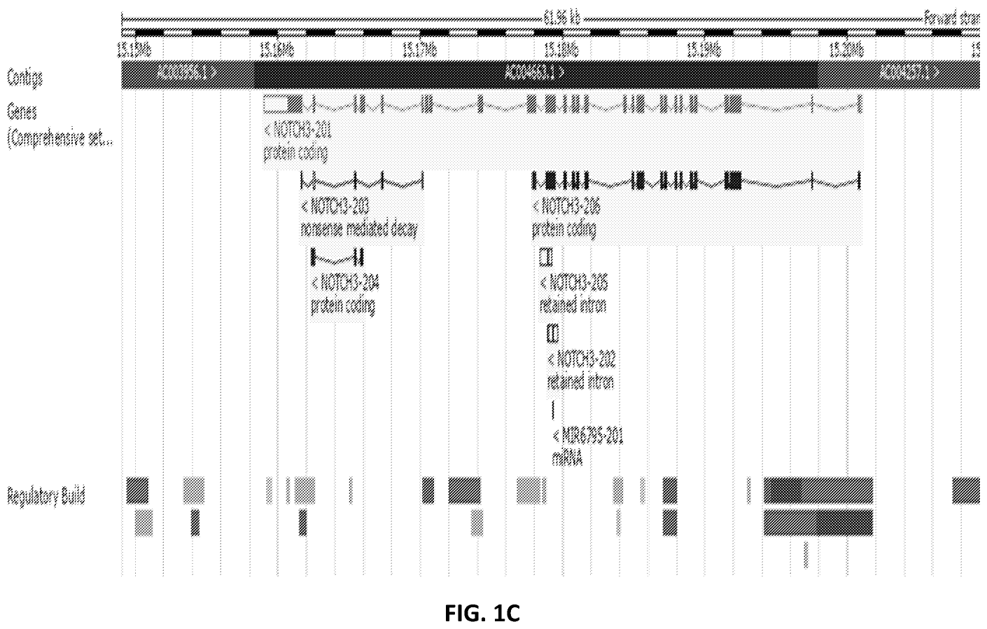

Aberrant NOTCH1 signaling, for example, related to activating NOTCH1 mutations and/or aberrant expression of the NOTCH1 ICD, is associated with a variety of cancers, including T cell acute lymphoblastic leukemias, or T-ALLs (see Weng et al., Science. 306(5694): 269-71, 2004; and Sulis et al., Blood. 112(3): 733-40, 2008), chronic lymphocytic leukemia (see Rosati et al., Front Oncol. 8:229, 2018), adenoid cystic carcinomas (see Ferrarotto et al., J Clin Oncol. 35(3):352-360, 2017), breast cancers (see Zhong et al., Onco Targets Ther. 9:6865-6871, 2016), and lung cancers such as non-small-cell lung carcinomas, or NSCLCs (see Westhoff et al., PNAS USA. 106(52):22293-22298, 2009), among others. In humans, the NOTCH1 gene is found on chromosome 9, at Chr 9: 136.49-136.55 Mb (see also FIG. 1A).

Some embodiments include treating a NOTCH3-associated cancer. Neurogenic locus notch homolog protein 3, or “NOTCH3”, is a human gene encoding a single-pass transmembrane heterodimer receptor protein for membrane-bound ligands Jagged1, Jagged2, and Delta1. In canonical NOTCH3 signaling, the intracellular domain (NICD3) travels to the nucleus, where it binds to CSL to replace the transcription repressors HDAC1 and silencing mediator for retinoid or thyroid-hormone receptors (SMRT) and recruits coactivators to activate transcription of Hey and Hes1, both of which are transcriptional regulators of the basic helix-loop-helix class. Notch3 expression has been found in vascular smooth muscle, the central nervous system, and subsets of thymocytes. Although the deletion of Notch1 and Notch2 is lethal to a developing embryo, deletion of Notch3 is not lethal. Despite sharing a similar basic structure with Notch1 and 2, Notch3 displays a number of structural differences. The most apparent difference is a missing transactivation domain (TAD), which could account for the weak transactivation activity of the NICD3. Slight differences are also evident in the Notch3 extracellular domain. For instance, it specifically lacks the Notch 1 and 2 equivalent of EGF repeat 21 and parts of EGF repeats 2 and 3. These differences likely relate to the differential ability of NICD3 to recruit coactivators or corepressors and undergo distinct conformational changes.

Aberrant NOTCH3 signaling, for example, related to activating NOTCH3 mutations and/or increased NOTCH3 gene copy number, is associated with a variety of cancers, including breast cancers such as triple negative breast cancers, T-ALLs, T-cell lymphoma, non-small cell lung cancers (NSCLCs), hemangioma, Kaposi's sarcoma, nasopharyngeal carcinoma, squamous cell carcinoma (SCC), ovarian cancer, colorectal cancer, hepatocellular carcinoma (HCC), and pancreatic cancers such as pancreatic ductal adenocarcinoma (PDAC) and prostatic adenocarcinoma (see, for example, Aburjania et al., Oncologist. 2018; 23(8):900-911, 2018), among others. In humans, the NOTCH3 gene is found on chromosome 19, at Chr 19: 15.16-15.2 Mb (see also FIG. 1B).

“YM155 monobromide” refers to the small molecule [1-(2-Methoxyethyl)-2-methyl-4,9-dioxo-3-(pyrazin-2-ylmethyl)-4,9-dihydro-1H-naphtho[2,3-d] imidazolium bromide], having the molecular formula C₂₀H₁₉N₄O₃·Br, and the CAS Number 781661-94-7, and includes pharmaceutically-acceptable salts and acids thereof. Also included are biologically-active or equivalent analogs and/or derivatives of YM155 monobromide, including prodrugs and pharmaceutically-acceptable salts thereof.

As noted above, certain embodiments comprising administering YM155 to the subject if ICN levels in the cancer tissue are increased relative to that of the control or reference, for example, wherein the control is a healthy tissue or reference derived from a healthy tissue. Typically, the term “intracellular NOTCH” or “ICN” refers to the cleaved and activated form of intracellular NOTCH, for example, the cleaved and activated form of intracellular NOTCH1 (ICN1), or the cleaved and activated form of intracellular NOTCH3 (ICN3). Certain embodiments comprise administering YM155 to the subject if the ICN levels in the cancer tissue are increased by a statistically significant amount relative to the ICN levels of the control or reference. Specific embodiments comprise administering YM155 to the subject if the ICN levels in the cancer tissue are increased by about or at least about 1.2, 1.3, 1.4, 1.5, 2, 3, 4, 5, 6, 7, 8, 9, or 10, 50, or 100-fold or more relative to the ICN levels of the control or reference. In some embodiments, the NOTCH is NOTCH1, and the ICN levels are ICN1 levels. In some embodiments, the NOTCH is NOTCH3, and the ICN levels are ICN3 levels.

ICN levels in a cancer tissue can be determined by any variety of methods. For example, ICN protein levels can be determined by immunohistochemistry (IHC) optionally chromogenic or fluorescent IHC, enzyme linked immunosorbent assay (ELISA), or Western blot on a human NOTCH1 protein or gene, among other assays. Certain embodiments thus include the step of determining or detecting or measuring ICN levels in a sample of cancer tissue from a subject in need thereof. Also included is the step of comparing the ICN levels in a sample of cancer tissue relative to that of a control or reference.

Certain embodiments include administering YM155 to the subject if the cancer tissue comprises an “activating” NOTCH mutation relative to wild-type NOTCH. In some instances, an “activating” NOTCH mutation includes at least one alteration (for example, substitutions, insertions, deletions) in the NOTCH protein sequence that lead to an increase in NOTCH signaling relative to wild-type NOTCH, and is associated with an increased risk of cancer or malignancy.

In some embodiments, the activating NOTCH mutation is an activating NOTCH1 mutation. General examples of activating NOTCH1 mutations include ligand-independent activation (LIA) NOTCH1 mutants and impaired degradation (ID) of NOTCH1 mutants. LIA NOTCH1 mutants lead to constitute activation of NOTCH1 signaling without ligand binding (see, for example, Weng et al., supra; Sulis et al., Blood. 112(3):733-740, 2008; Asworth et al., Blood. 116(25):5455-5464, 2010; Haydu et al., Blood. 119(22):5211-5214, 2012; and Ellisen et al., 1991; 66(4):649-661, 1991). ID of NOTCH1 mutants relate to the observation that NOTCH1 transcriptional activation is terminated by the proteasomal degradation of the NOTCH1 ICD, which is induced by the proline, glutamic acid, serine, threonine—rich (PEST) domain recognition of the FBXW7-SCF ubiquitin ligase complex; here, alterations in the C-terminal PEST domain of NOTCH1 (and optionally FBXW7) result in ID of the NOTCH1 ICD, leading to aberrantly prolonged NOTCH1 signaling (see, for example, Thompson et al., J Exp Med. 204(8):1825, 2007; O'Neil et al., J Exp Med. 204(8):1813-1824, 2007; and Puentes et al., Nature. 526(7574):519-524, 2015). Particular examples of activating NOTCH1 mutations include heterodimerization domain (HD) mutants, including single nucleotide polymorphisms (SNP) in the HD or small in-frame insertions or deletions in the HD, juxtamembrane expansion (JME) alleles, and C-terminal PEST domain mutants, among others (see the foregoing references).

In some embodiments, the activating NOTCH mutation is an activating NOTCH3 mutation. Examples of activating NOTCH3 mutations include C-terminal Pro-Glu-Ser-Thr-rich (PEST) domain mutants (see, for example, Wang et al., Clin Cancer Res. 21:1487-1496, 2015; and Kawazu et al., PLoS Genet. 13:e1006853, 2017), among others.

NOTCH mutation status in the cancer tissue can be determined by any variety of methods. For instance, in some embodiments, NOTCH mutation status is determined by in situ hybridization (ISH), fluorescence in situ hybridization (FISH), whole exome sequencing (WES), single nucleotide polymorphism (SNP) array, next generation sequencing (NGS), or comparative genome hybridization (CGH) on a human NOTCH1 protein or gene. Thus, the step of determining NOTCH mutation status, for example, to identify activating NOTCH (e.g., NOTCH1, NOTCH3) mutations of interest, can be performed according to routine techniques in the art.

Certain embodiments include administering YM155 to the subject if the NOTCH gene copy number in the cancer tissue is increased relative to that of a NOTCH copy gene reference, for example, the NOTCH1 or NOTCH3 gene copy number. In certain embodiments, the NOTCH gene copy number in the sample of cancer tissue is increased by about or at least about 1.5, 2, 3, 4, 5, 6, 7, 8, 9, or 10-fold relative to that of the NOTCH gene copy number reference. The NOTCH gene copy number in the cancer tissue can be determined by any variety of methods. For instance, in some embodiments, NOTCH gene copy number is determined by array comparative genome hybridization (aCGH), single nucleotide polymorphism (SNP) array, copy number variation (CNV) sequencing, or multiplex ligation-dependent probe amplification (MLPA). Some embodiments include obtaining the NOTCH gene copy number reference, for example, the NOTCH1 or NOTCH3 gene copy number reference, from a database, or determining the NOTCH gene copy number reference from a non-cancerous tissue from a control.

CGH refers to a molecular cytogenetic method for analyzing copy number variations (CNVs) relative to ploidy level in the DNA of a test sample compared to a reference sample, without the need for culturing cells. This technique allows quick and efficient comparisons between two genomic DNA samples arising from two sources, which are most often closely related, because it is suspected that they contain differences in terms of either gains or losses of either whole chromosomes or subchromosomal regions (a portion of a whole chromosome). The technique was originally developed for the evaluation of the differences between the chromosomal complements of solid tumor and normal tissue (see, e.g., Kallioniemi et al., Science. 258 (5083): 818-821, 1992). The use of DNA microarrays in conjunction with CGH techniques has led to the development of a more specific form of array CGH (aCGH), allowing for a locus-by-locus measure of CNV with increased resolution as low as 100 kilobases (see, e.g., Pinkel, Annu Rev Genom Hum Genet. 6:331-354, 2005). CNV is a prevalent form of critical genetic variation that leads to an abnormal number of copies of large genomic regions in a cell, and high-resolution sequence data can be analyzed by next-generation sequencing (NGS) to identify the same (see, e.g., Zhao et al., BMC Bioinformatics. 14 Suppl 11:S1, 2013). MLPA refers to a variation of the multiplex polymerase chain reaction that permits amplification of multiple targets with only a single primer pair (see, e.g., Schouten et al., Nucleic Acids Res. 30 (12): e57, 2002). In situ hybridization (ISH) and fluorescent in situ hybridization (FISH) refer to a type of hybridization that uses a labeled complementary DNA, RNA or modified nucleic acids strand (i.e., probe) to localize a specific DNA or RNA sequence in a portion or section of tissue (in situ) (see, e.g., Parra & Windle, Nature Genetics. 5:17-21, 1993; and Gall & Pardue, PNAS USA. 63: 378-383, 1969). Thus, in some instances, the methods and kits described herein employ any one or more of the foregoing techniques and/or comprise reagents for performing the same.

Examples of a “reference” include a value, amount, sequence, or other characteristic obtained from a database, for example, a “wild-type” NOTCH sequence (see, e.g., FIG. 1A and Ensembl:ENSG00000148400 for human NOTCH1 gene references; and FIG. 1B and ENSG00000074181 for human NOTCH3 gene references). A “reference” also includes value, amount, sequence, or other characteristic obtained from a non-cancerous tissue from one or more controls, for example, one or more healthy or non-cancerous control subjects (e.g., a population of healthy or non-cancerous control subjects), or one or more corresponding non-cancerous control tissues from the subject being tested. Typically, a “corresponding” non-cancerous control tissue is obtained from the same type of tissue as the cancer tissue being tested. As with the cancer tissue, the ICN levels from a non-cancerous control can be determined by any variety of methods, including, for example, by IHC, for example, chromogenic or fluorescent IHC, ELISA, or Western blot on a human NOTCH protein, for example, NOTCH1 or NOTCH3 (supra) Similarly, the NOTCH mutation status from a non-cancerous control can be determined by any variety of methods, including, for example, ISH, FISH, WES, SNP array, NGS, or CGH on a human NOTCH protein or gene, for example, a human NOTCH1 or NOTCH3 protein or gene (supra). NOTCH gene copy number from a non-cancerous control can be determined by any variety of methods, including, for example, aCGH, SNP array, CNV sequencing, or MLPA.

In some embodiments, the sample of cancer tissue (or non-cancerous control tissue) is a blood sample, a surgical sample, a biopsy sample, a pleural effusion sample, or an ascetic fluid sample from the subject. Particular examples of samples of cancer tissues (or non-cancerous control tissues) include white blood cell, lung, breast, gastrointestinal (stomach, colon, rectal), ovarian, pancreatic, liver, bladder, cervical, neuronal, uterine, salivary gland, kidney, prostate, thyroid, or muscle tissues. Certain embodiments include the step of obtaining the sample of cancer tissue (or non-cancerous control tissue) from the subject, for example, prior to determining ICN levels, NOTCH mutation status, and/or NOTCH gene copy number.

In some embodiments, the subject is a human subject.

As noted above, certain embodiments include administering to the subject an anti-cancer agent excluding (or other than) YM155 monobromide if the subject is characterized as non-responsive to YM155 monobromide therapy, for example, if (i) ICN levels in the cancer tissue are not substantially increased or undetectable relative to the control or reference, (ii) if the cancer tissue lacks an activating NOTCH mutation relative to wild-type NOTCH, and/or (iii) if NOTCH gene copy number is not substantially increased relative to that of the NOTCH copy gene reference. Exemplary anti-cancer agents (other than YM155 monobromide) for administering to a subject characterized as non-responsive to YM155 monobromide therapy include immunotherapy agents, chemotherapeutic agents, hormonal therapeutic agents, and/or kinase inhibitors, as described herein and known in the art.

Examples of chemotherapeutic agents include alkylating agents, anti-metabolites, cytotoxic antibiotics, topoisomerase inhibitors (type 1 or type II), and anti-microtubule agents, among others.

Examples of alkylating agents include nitrogen mustards (e.g., mechlorethamine, cyclophosphamide, mustine, melphalan, chlorambucil, ifosfamide, and busulfan), nitrosoureas (e.g., N-Nitroso-N-methylurea (MNU), carmustine (BCNU), lomustine (CCNU), semustine (MeCCNU), fotemustine, and streptozotocin), tetrazines (e.g., dacarbazine, mitozolomide, and temozolomide), aziridines (e.g., thiotepa, mytomycin, and diaziquone (AZQ)), cisplatins and derivatives thereof (e.g., carboplatin and oxaliplatin), and non-classical alkylating agents (optionally procarbazine and hexamethylmelamine).

Examples of anti-metabolites include anti-folates (e.g., methotrexate and pemetrexed), fluoropyrimidines (e.g., 5-fluorouracil and capecitabine), deoxynucleoside analogues (e.g., ancitabine, enocitabine, cytarabine, gemcitabine, decitabine, azacitidine, fludarabine, nelarabine, cladribine, clofarabine, fludarabine, and pentostatin), and thiopurines (e.g., thioguanine and mercaptopurine);

Examples of cytotoxic antibiotics include anthracyclines (e.g., doxorubicin, daunorubicin, epirubicin, idarubicin, pirarubicin, aclarubicin, and mitoxantrone), bleomycins, mitomycin C, mitoxantrone, and actinomycin. Examples of topoisomerase inhibitors include camptothecin, irinotecan, topotecan, etoposide, doxorubicin, mitoxantrone, teniposide, novobiocin, merbarone, and aclarubicin.

Examples of anti-microtubule agents include taxanes (e.g., paclitaxel and docetaxel) and vinca alkaloids (e.g., vinblastine, vincristine, vindesine, vinorelbine).

General examples of hormonal therapeutic agents include hormonal agonists and hormonal antagonists. Particular examples of hormonal agonists include progestogen (progestin), corticosteroids (e.g., prednisolone, methylprednisolone, dexamethasone), insulin like growth factors, VEGF derived angiogenic and lymphangiogenic factors (e.g., VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, androgens, estrogens, and somatostatin analogs. Examples of hormonal antagonists include hormone synthesis inhibitors such as aromatase inhibitors and gonadotropin-releasing hormone (GnRH)s agonists (e.g., leuprolide, goserelin, triptorelin, histrelin) including analogs thereof. Also included are hormone receptor antagonist such as selective estrogen receptor modulators (SERMs; e.g., tamoxifen, raloxifene, toremifene) and anti-androgens (e.g., flutamide, bicalutamide, nilutamide).

Also included are hormonal pathway inhibitors such as antibodies directed against hormonal receptors. Examples include inhibitors of the IGF receptor (e.g., IGF-IR1) such as cixutumumab, dalotuzumab, figitumumab, ganitumab, istiratumab, and robatumumab; inhibitors of the vascular endothelial growth factor receptors 1, 2 or 3 (VEGFR1, VEGFR2 or VEGFR3) such as alacizumab pegol, bevacizumab, icrucumab, ramucirumab; inhibitors of the TGF-beta receptors R1, R2, and R3 such as fresolimumab and metelimumab; inhibitors of c-Met such as naxitamab; inhibitors of the EGF receptor such as cetuximab, depatuxizumab mafodotin, futuximab, imgatuzumab, laprituximab emtansine, matuzumab, modotuximab, necitumumab, nimotuzumab, panitumumab, tomuzotuximab, and zalutumumab; inhibitors of the FGF receptor such as aprutumab ixadotin and bemarituzumab; and inhibitors of the PDGF receptor such as olaratumab and tovetumab.

Examples of kinase inhibitors include, without limitation, adavosertib, afanitib, aflibercept, axitinib, bevacizumab, bosutinib, cabozantinib, cetuximab, cobimetinib, crizotinib, dasatinib, entrectinib, erdafitinib, erlotinib, fostamitinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemuafenib.

The methods described herein can be used in the treatment and/or diagnosis of any variety of NOTCH-associated cancers or tumors. In some embodiments, the cancer is a primary cancer, i.e., a cancer growing at the anatomical site where tumor progression began and yielded a cancerous mass. In some embodiments, the cancer is a secondary or metastatic cancer, i.e., a cancer which has spread from the primary site or tissue of origin into one or more different sites or tissues. In some instances, the cancer is selected from one or more of a leukemia, lymphoma (including Hodgkin's lymphoma and Non-Hodgkin's lymphoma), carcinoma, sarcoma such as rhabdomyosarcoma for example, alveolar rhabdomyosarcoma, (including sarcoma originating in the bones, tendons, cartilage, muscle, fat, fibrous, blood vessels, adipose, and/or connective tissue), breast cancer (including metastatic breast cancer), lung cancer (including non-small cell lung cancer, small cell lung cancer, adenocarcinoma, and squamous carcinoma of the lung), neuroblastoma, medulloblastoma, astrocytoma, glioblastoma multiforme, retinoblastoma, myeloma, adenosquamous carcinoma, carcinosarcoma, mixed mesodermal tumor, teratocarcinoma), gastrointestinal cancer, stomach cancer, colorectal cancer, colon cancer, rectal cancer, ovarian cancer, pancreatic cancer, liver cancer, bladder cancer, cervical cancer, glioblastoma, uterine carcinoma, salivary gland carcinoma, kidney or renal cancer (e.g., Wilm's tumor), prostate cancer, thyroid cancer, and head and neck cancer.

In specific embodiments, the NOTCH-associated cancer is a leukemia or lymphoma, for example, a T-cell lymphoblastic leukemia (T-ALL), a T-cell lymphoblastic lymphoma (T-LBL), or chronic lymphocytic leukemia (CLL). In some embodiments, the NOTCH-associated cancer is a non small cell lung cancer or a breast cancer, including triple-negative breast cancer. In specific embodiments, the T-ALL is a NOTCH1-associated T-ALL, or a NOTCH3-associated T-ALL. In particular embodiments, the breast cancer is NOTCH-1 associated breast cancer, or a NOTCH3-associated breast cancer, for example, a triple-negative breast cancer (TNBR).

In certain embodiments, the methods described herein are sufficient to result in tumor regression, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the methods described are sufficient to result in stable disease. In certain embodiments, the methods described herein are sufficient to result in clinically relevant reduction in symptoms of a particular disease indication known to the skilled clinician.

The methods for treating cancers can be combined with other therapeutic modalities. For example, a combination therapy described herein can be administered to a subject before, during, or after other therapeutic interventions, including symptomatic care, radiotherapy, surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Methods for identifying subjects with one or more of the diseases or conditions described herein are known in the art.

For in vivo use, for instance, for the treatment of human disease or testing, the agents described herein are generally incorporated into one or more therapeutic or pharmaceutical compositions prior to administration.

To prepare a therapeutic or pharmaceutical composition, an effective or desired amount of one or more agents is typically mixed with any pharmaceutical carrier(s) or excipient known to those skilled in the art to be suitable for the particular agent and/or mode of administration. A pharmaceutical carrier may be liquid, semi-liquid or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

Administration of agents described herein, in pure form or in an appropriate therapeutic or pharmaceutical composition, can be carried out via any of the accepted modes of administration of agents for serving similar utilities. The therapeutic or pharmaceutical compositions can be prepared by combining an agent-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other small molecules as described elsewhere herein) and/or suitable excipients such as salts, buffers and stabilizers may, but need not, be present within the composition.

Administration may be achieved by a variety of different routes, including oral, parenteral, nasal, intravenous, intradermal, intramuscular, subcutaneous or topical. Preferred modes of administration depend upon the nature of the condition to be treated or prevented. Particular embodiments include administration by IV infusion.

Carriers can include, for example, pharmaceutically- or physiologically-acceptable carriers, excipients, or stabilizers that are non-toxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In some embodiments, one or more agents can be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related therapeutic or pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Therapeutic or pharmaceutical compositions according to certain embodiments of the present disclosure are formulated so as to allow the active ingredients contained therein to be bioavailable upon administration of the composition to a subject or patient. Compositions that will be administered to a subject or patient may take the form of one or more dosage units, where for example, a tablet may be a single dosage unit, and a container of a herein described agent in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will typically contain a therapeutically effective amount of an agent described herein, for treatment of a disease or condition of interest.

A therapeutic or pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid. Certain embodiments include sterile, injectable solutions.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The therapeutic or pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid therapeutic or pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid therapeutic or pharmaceutical composition intended for either parenteral or oral administration should contain an amount of an agent such that a suitable dosage will be obtained. Typically, this amount is at least 0.01% of the agent of interest in the composition. When intended for oral administration, this amount may be varied to be between 0.1 and about 70% of the weight of the composition. Certain oral therapeutic or pharmaceutical compositions contain between about 4% and about 75% of the agent of interest. In certain embodiments, therapeutic or pharmaceutical compositions and preparations according to the present invention are prepared so that a parenteral dosage unit contains between 0.01 to 10% by weight of the agent of interest prior to dilution.

The therapeutic or pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The therapeutic or pharmaceutical compositions in solid or liquid form may include a component that binds to agent and thereby assists in the delivery of the compound. Suitable components that may act in this capacity include monoclonal or polyclonal antibodies, one or more proteins or a liposome.

The compositions described herein may be prepared with carriers that protect the agents against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The therapeutic or pharmaceutical compositions may be prepared by methodology well known in the pharmaceutical art. For example, a therapeutic or pharmaceutical composition intended to be administered by injection may comprise one or more of salts, buffers and/or stabilizers, with sterile, distilled water so as to form a solution. A surfactant may be added to facilitate the formation of a homogeneous solution or suspension. Surfactants are compounds that non-covalently interact with the agent so as to facilitate dissolution or homogeneous suspension of the agent in the aqueous delivery system.

Certain embodiments include the use of a diagnostic kit for determining or predicting a therapeutic response (or responsiveness) to YM155 monobromide [1-(2-Methoxyethyl)-2-methyl-4,9-dioxo-3-(pyrazin-2-ylmethyl)-4,9-dihydro-1H-naphtho[2,3-d] imidazolium bromide] therapy in a subject with cancer, comprising means for determining or measuring (i) intracellular NOTCH (ICN) levels, (ii) NOTCH mutation status, and/or (iii) NOTCH gene copy number, in a sample of cancer tissue from the subject, including cancer tissue and non-cancerous tissue. Also included are patient care kits, comprising: (a) means for determining or measuring (i) intracellular NOTCH (ICN) levels, (ii) NOTCH mutation status, and/or (iii) NOTCH gene copy number, in a sample of cancer tissue from the subject, including cancer tissue and non-cancerous tissue; and (b) YM155 monobromide [1-(2-Methoxyethyl)-2-methyl-4,9-dioxo-3-(pyrazin-2-ylmethyl)-4,9-dihydro-1H-naphtho[2,3-d] imidazolium bromide]. In certain embodiments, the NOTCH is NOTCH1 or NOTCH3.

In some embodiments, the means for determining or measuring ICN levels comprise reagents for performing a diagnostic assay selected from one or more of IHC, for example, chromogenic or fluorescent IHC, ELISA, or Western blot on a human NOTCH protein, for example, NOTCH1 or NOTCH3. In some embodiments, the means for determining NOTCH mutation status, for example, NOTCH1 or NOTCH3 mutation status, comprise reagents for performing a diagnostic assay selected from one or more of ISH, FISH, WES, SNP array, NGS, or CGH on a human NOTCH protein or gene. In some embodiments, the means for determining or measuring NOTCH gene copy number, for example, NOTCH1 or NOTCH3 gene copy number, in the sample of cancer tissue by aCGH, SNP array, CNV sequencing, or MLPA.

Some diagnostic or patient care kits include a NOTCH gene reference obtained from a database, or determined from a non-cancerous tissue from a control or reference. The kits can also include written instructions, for example, on how to determine or measure ICN levels, NOTCH mutation status, and/or NOTCH gene copy number in a sample of cancer tissue from a subject, and/or from a non-cancerous control.

In some embodiments, a diagnostic or patient care kit contains separate containers, dividers, or compartments for the composition(s) and informational material(s). For example, the composition(s) or reagents can be contained in a bottle, vial, or syringe, and the informational material(s) can be contained in association with the container. In some embodiments, the separate elements of the kit are contained within a single, undivided container. For example, the composition(s) or reagents are contained in a bottle, vial or syringe that has attached thereto the informational material in the form of a label. In some embodiments, the kit includes a plurality (e.g., a pack) of individual containers, each containing one or more compositions, reagents, and/or unit dosage forms of YM155 monobromide. For example, the kit includes a plurality of syringes, ampules, foil packets, or blister packs, each containing a reagent or a single unit dose of YM155 monobromide. The containers of the kits can be air tight, waterproof (e.g., impermeable to changes in moisture or evaporation), and/or light-tight.

The patient care kit optionally includes a device suitable for administration of the agent(s), e.g., a syringe, inhalant, dropper (e.g., eye dropper), swab (e.g., a cotton swab or wooden swab), or any such delivery device. In some embodiments, the device is an implantable device that dispenses metered doses of the agent(s). Also included are methods of providing a kit, e.g., by combining the components described herein.

In certain aspects, the diagnostic or therapeutic response tests or methods described herein are performed at a diagnostic laboratory, and the results are then provided to the subject, or to a physician or other healthcare provider that plays a role in the subject's healthcare and cancer treatment. Particular embodiments thus include methods for providing the results of the responsiveness test to the subject in need thereof, or to the physician or other healthcare provider. These results or data can be in the form of a hard-copy or paper-copy, or an electronic form, such as a computer-readable medium.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill certain changes and modifications may be made thereto without departing from the spirit or scope of the description or appended claims. The following examples are provided by way of illustration only and not by way of limitation. Those of skill in the art will readily recognize a variety of noncritical parameters that could be changed or modified to yield essentially similar results.

### EXAMPLES

### Example 1

### Evaluation of YM155 Sensitivity in Human T-ALL Cells

Experiments were performed to evaluate the sensitivity human T-lineage acute lymphoblastic leukemia (T-ALL) cancer cells to treatment with YM155, and also to evaluate NOTCH1 status in relation to that sensitivity.

Cell Culture. CCRF-CEM, Jurkat, MOLT-4, and LOUCY cells were cultured in RPMI1640 (Hyclone™, USA) supplemented with 10% fetal bovine serum (Gibco, USA). 12.1 (CRL-2572) cells were cultured in RPMI1640 (Hyclone™, USA) supplemented with 15% fetal bovine serum (Gibco, USA) with Hepes. All cells were cultured in humidified incubator with 5% CO₂ at 37° C.

CCRF-CEM cells were purchased from Cell Bank, Shanghai Institutes for Biological Sciences, Chinese Academy of Sciences (Shanghai, China). MOLT-4, 12.1 (CRL-2572) and Jurkat cells were purchased from National Infrastructure of Cell Line Resource (Beijing, China). Loucy cells were purchased from American Type Culture Collection (USA).

Cell Treatment and proliferation assay. CCRF-CEM, Jurkat, MOLT-4, 12.1 (CRL-2572), and LOUCY cells were seeded in 96-well plates (Corning, USA) at 20000 cells/well. Cells were treated with YM155 (200, 100, 50, 25, 12.5, 6.25, 3.125, 1.5625, 0.78125 nM APExBIO, A4221) or 0.1% DMSO (VWR, USA). After 72 hours incubation, cell proliferation was measured by XTT assay (Cell Proliferation Kit II XTT, sigma, 11465015001) according to protocols. Briefly, after YM155 treatment, added 50 μl XTT labeling mixture to each well then incubated the microplate for 4 hours in humidified incubator with 5% CO₂ at 37° C. The absorbance of wavelength at 490 nm (OD₄₉₀) and 650 nm (OD₆₅₀) were detected respectively by SpectraMax 190. The IC₅₀ was calculated by the value of OD₄₉₀-OD₆₅₀.

As shown in FIG. 1, YM155 inhibits cell proliferation of human T-lineage acute lymphoblastic leukemia cell. However, the proliferation inhibitory effects of YM155 were different in these cell lines, and correlated with NOTCH1 status. Cells (CCRF-CEM, Jurkat, MOLT-4 and 12.1) with activating NOTCH1 were more sensitive to YM155 than cells (LOUCY) with wild-type (WT) NOTCH1 (see Table E1).

TABLE E1
NOTCH1 mutation status and YM155
IC₅₀ in human T-ALL cell lines

| | CCRF- | | | 12.1 (CRL- | |
|---|---|---|---|---|---|
| | CEM | Jurkat | MOLT-4 | 2572) | LOUCY |
| IC₅₀ (nM) | 19.17 | 23.63 | 6.31 | 6.48 | 90.4 |
| NOTCH1 | Activating | Activating | Activating | Activating | WT |
| status | mutation | mutation | mutation | mutation | |

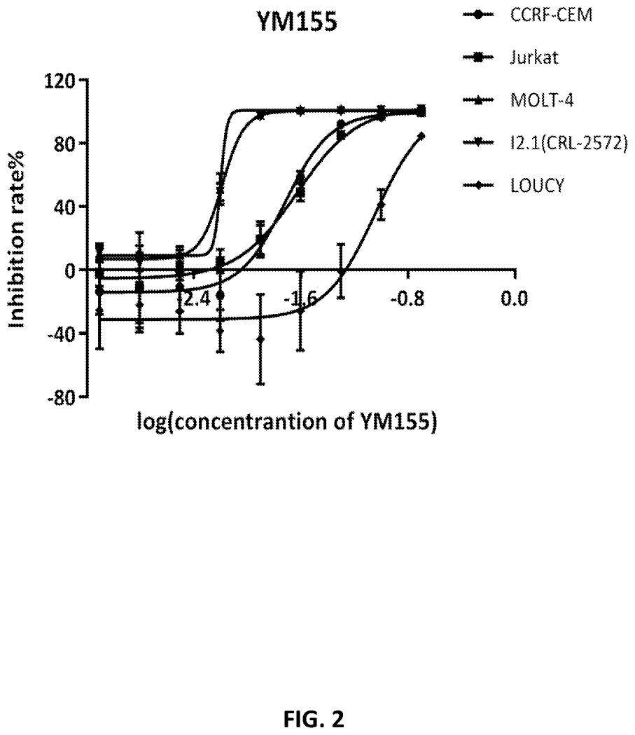

Western Blot. CCRF-CEM, Jurkat, MOLT-4, 12.1 (CRL-2572), and LOUCY cells were harvested and centrifuged at 200 g for 5 minutes to obtain cell pellets. Then the pellets were lysed in lysis buffer (Beyotime, China) which additionally added with protease inhibitor cocktail (Beyotime, China) and phosphatase inhibitor cocktail (Beyotime, China). Cells were incubated on ice for 30 minutes and then centrifuged at 4° C., 12,000 rpm for 10 minutes to obtain the supernatant as cell lysate. The concentrations of protein in cell lysate were determined by Micro BCA™ Protein Assay Kit (ThermoFisher, USA). 5×SDS-PAGE Sample Loading Buffer (Beyotime, China) was added into cell lysate contained 40 μg of total protein and, after boiling, the mixture was electrophoresed in polyacrylamide gel. After electrophoresis, proteins on the gel were transferred to PVDF membrane, and the membrane was cut at position close to the molecular weights of proteins whose expression was examined (Cleaved Notch1 (CST, USA), GAPDH (ZSGB-BIO, China)). FIG. 2 shows the Western blot analysis of activated NOTCH1 (ICN1) in CCRF-CEM, Jurkat, MOLT-4, I2.1 (CRL-2572), and LOUCY cells. GAPDH is shown as loading control.

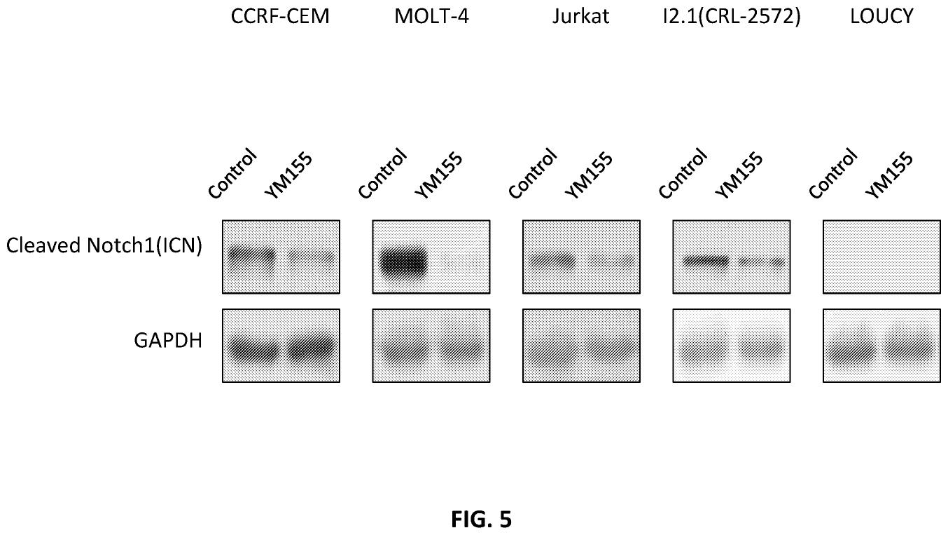

CCRF-CEM, Jurkat, MOLT-4, 12.1 (CRL-2572), and LOUCY cells were also treated with YM155 for 72 hours (0.1% DMSO was added as control), harvested, and centrifuged at 200 g for 5 minutes to obtain cell pellets. Then the pellets were lysed in lysis buffer (Beyotime, China) which additionally added with protease inhibitor cocktail (Beyotime, China) and phosphatase inhibitor cocktail (Beyotime, China). Cells were incubated on ice for 30 minutes and then centrifuged at 4° C., 12000 rpm for 10 minutes to obtain the supernatant as cell lysate. The concentrations of protein in cell lysate were determined by Micro BCA™ Protein Assay Kit (ThermoFisher, USA). 5×SDS-PAGE Sample Loading Buffer (Beyotime, China) was added into cell lysate contained 40 μg of total protein and, after boiling, the mixture was electrophoresed in polyacrylamide gel. After electrophoresis, proteins on the gel were transferred to PVDF membrane, and the membrane was cut at position close to the molecular weights of proteins whose expression was examined (Cleaved Notch1 (CST, USA), GAPDH (ZSGB-BIO, China)). The Western blot in FIG. 5 shows that YM155 treatment reduced the levels of activated NOTCH1 (ICN1) in CCRF-CEM, Jurkat, MOLT-4, and I2.1 (CRL-2572) cells relative to control; as above, LOUCY cells were negative for ICN1.

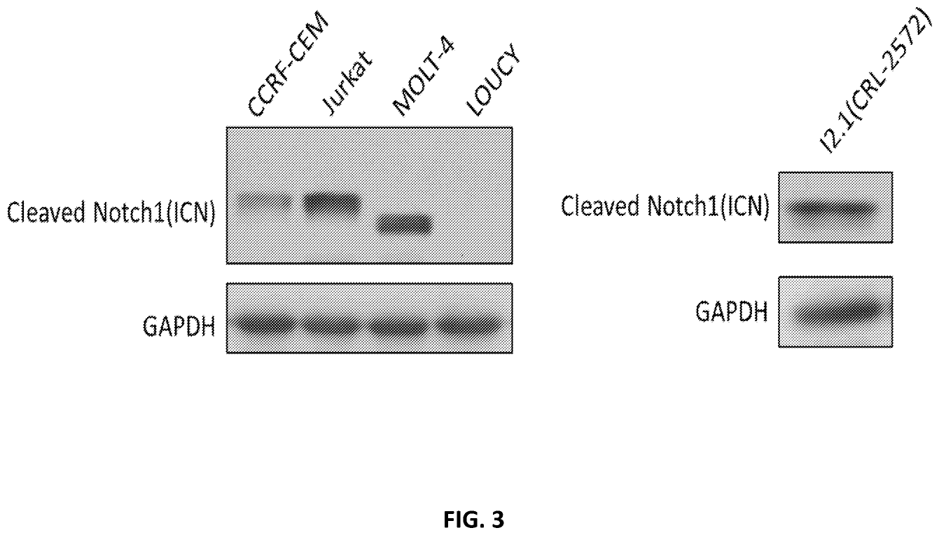

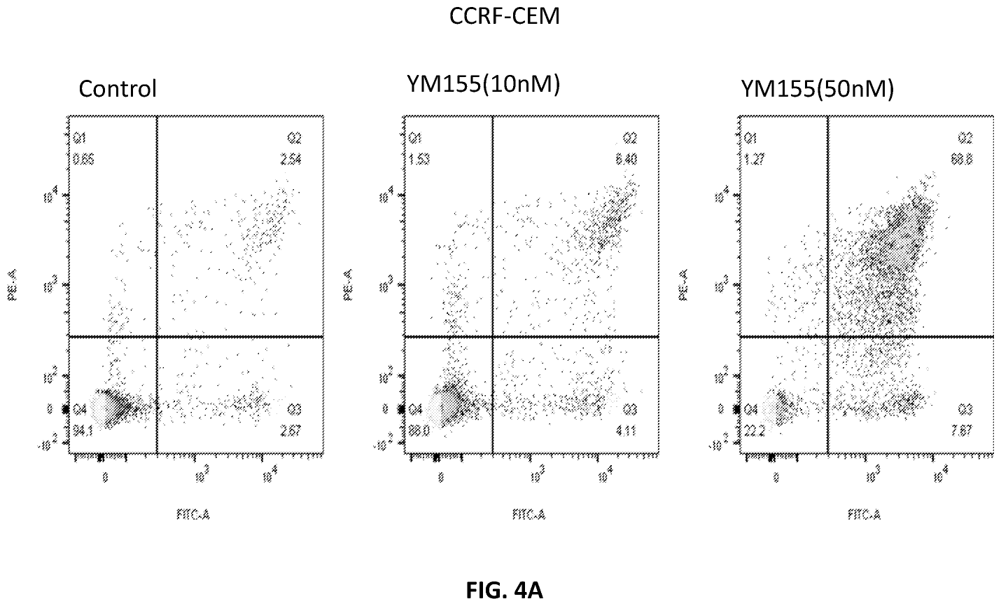

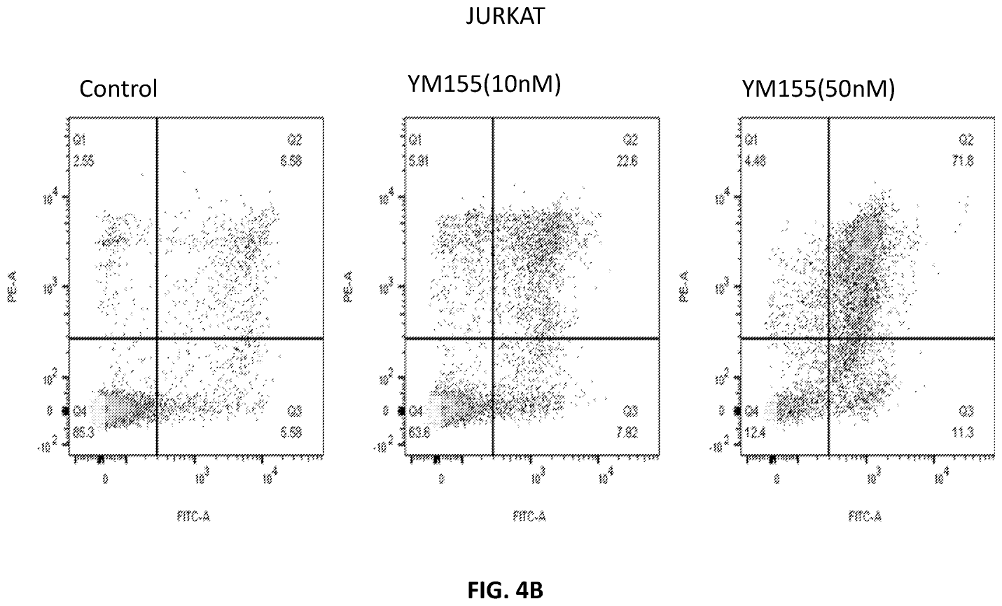

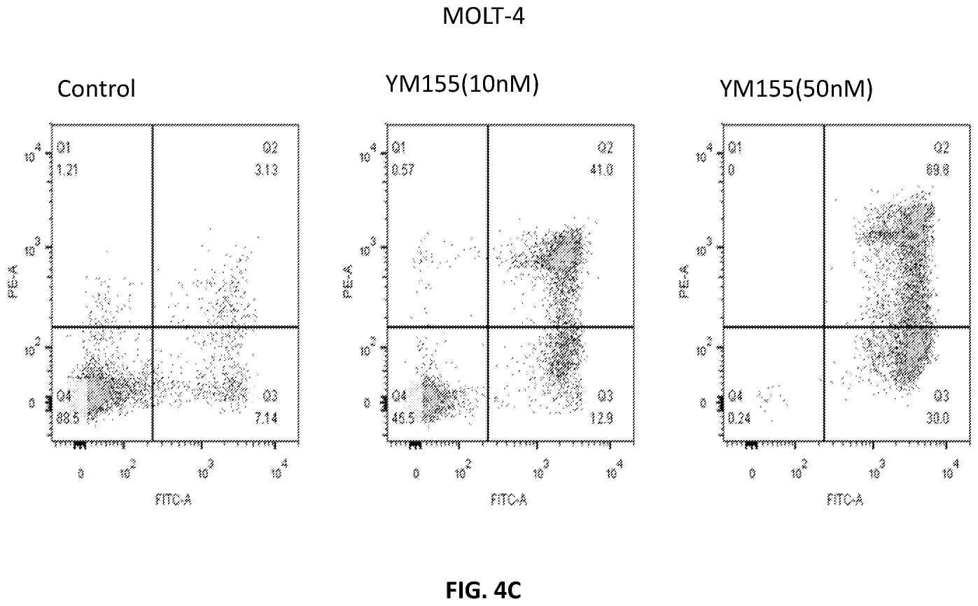

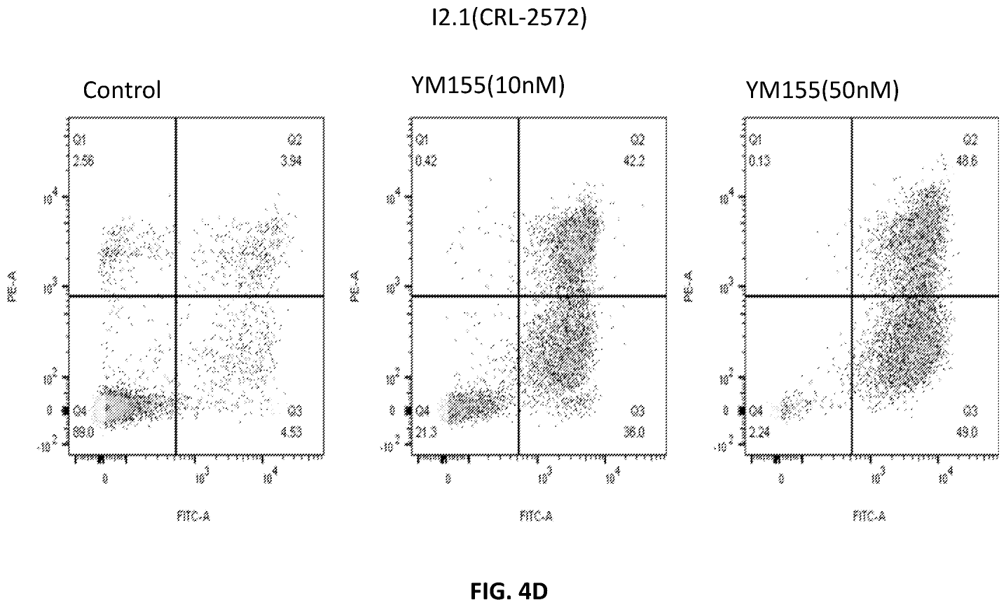

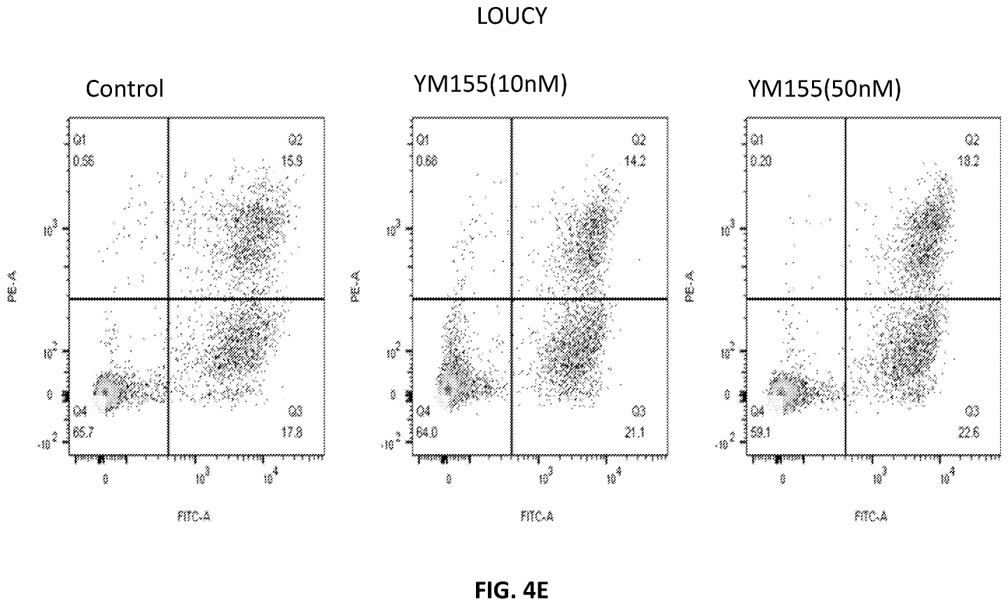

Flow Cytometry. CCRF-CEM, Jurkat, MOLT-4, 12.1 (CRL-2572), and LOUCY cells were treated with YM155 (10 nM, 50 nM) for 72 hours (0.1% DMSO was added as control). Cells were harvested and fixed with 70% cold ethanol (FUCHEN, China). Ethanol-fixed cells were stained with propidium iodide (PI) and Annexin V using apoptosis detection kits (Thermo) and analyzed on a BD FACS Analyser (LSRFortessa). Data were analyzed using FlowJo software. As shown in FIGS. 3A-3E, YM155 induced significantly higher levels of apoptosis in NOTCH1-associated cancer cells (FIGS. 3A-3D) relative to LOUCY cells that are characterized by wild-type NOTCH1.

### Example 2

### Evaluation of YM155 Sensitivity in Human Breast Cancer Cells

Experiments were performed to evaluate the sensitivity human breast cancer cells to treatment with YM155, and also to evaluate NOTCH1 status in relation to that sensitivity.

Cell culture. The cell lines used in the study were breast cancer cell lines HCC1143, AU565, T47D, and MDA-MB-157. MDA-MB-157 was acquired from National Infrastructure of Cell Line Resource (Beijing, China). HCC1143 and AU565 were obtained from Cobioer Biosciences (Nanjing, China). T47D was purchased from The Cell Bank of Type Culture Collection of Chinese Academy of Sciences, Shanghai, China. MDA-MB-157, HCC1143 and AU565 were maintained in HyClone™ RPMI1640 (Cytiva, USA) supplemented with 10% Gibco™ fetal bovine serum (FBS, Fisher Scientific, USA). T47D was cultured in DMEM (ThermoFisher, USA) supplemented with 10% FBS.

Cell proliferation assay. The cells were seeded in 96-well plates (Corning, USA) at 2×10⁴ cells/well. Cells were treated with YM155 (APExBIO, A4221) at designated concentrations (200, 100, 50, 25, 12.5, 6.25, 3.125, 1.5625, 0.78125 nM, respectively) or 0.1% DMSO (VWR, USA). After 72 hours incubation, cell proliferation was measured by MTS assay method (CellTiter 96® Aqueous Non-Radioactive Cell Proliferation Assay, Promega, USA) according to manufacturer's instruction. Briefly, freshly prepared MTS/PMS solution was pipetted into each well of the 96-well assay plate containing 100 μl of cells in culture medium. After incubation for 1-4 hours at 37° C. and 5% CO₂, the absorbance of the mixtures was measured at 490 nm using an ELISA plate reader. IC₅₀ values were calculated from the absorbance curve at various drug concentrations.

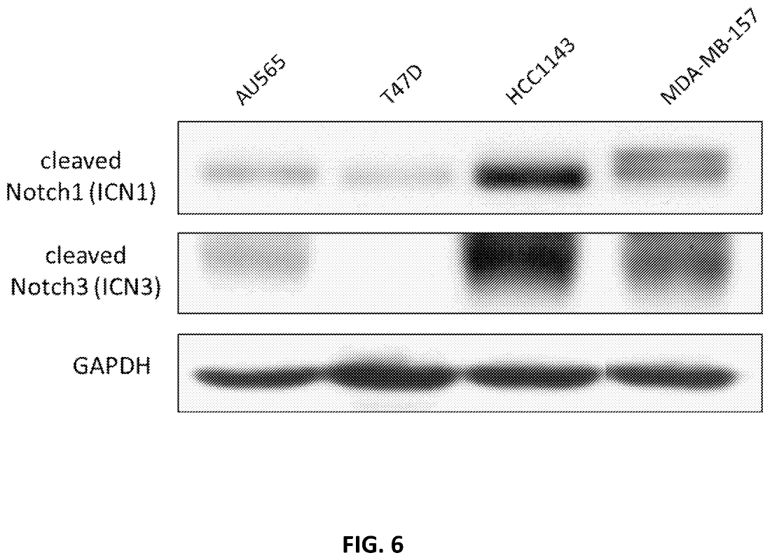

Western Blot. The cells grown to 70-80% confluency were harvested and lysed in a lysis buffer containing protease inhibitor cocktail (Beyotime, China) at 4° C. The concentrations of total protein in the cell lysates were determined by BCA Protein Assay Kit (ThermoFisher, USA). Forty micrograms of total protein was used in each lane for SDS-PAGE and western-blot assay. Antibodies against cleaved Notch1 and Notch3 (Cell Signaling, USA) were used to detect endogenous levels of the Notch1 and Notch3 intracellular domain (ICN1 and ICN3). FIG. 6 shows a Western blot analysis of intracellular cleaved and activated NOTCH1 (ICN1) and NOTCH3 (ICN3) in breast cancer cell lines, specifically AU656, T47D, HCC1143, and MDA-MB-157 cells. HCC1143 cells expressed the highest levels of ICN1/ICN3, followed by MDA-MB-157 cells. AU565 cells expressed lower levels of ICN1 and ICN3, and T47D expressed similarly low levels of ICN1 but no detectable ICN3.

Apoptosis assay. Cellular apoptosis was assayed with Alexa Flour™ 488 Annexin V/Dead cell apoptosis Kit (ThermoFisher, USA) and GreenNuc™ live cell caspase 3 activity assay kit (Beyotime Biotechnology, China). Briefly, cells cultured in 6-well tissue culture plates were treated with YM155, DMSO, and mitomycin C (positive control), respectively. After 1-4 day incubation, the cells were collected and assayed for apoptosis according to manufacturers' instructions. For Annexin V assay, the cell suspensions were incubated with Alexa Flour™ 488 labeled Annexin V and Propidium Iodide (PI). For the caspase 3 activity assay, a caspase 3 substrate was added to generate fluorescent product. The cells were then analyzed on a BD LSRFortessa cell analyzer. Flow cytometry data was analyzed with Flowjo software.

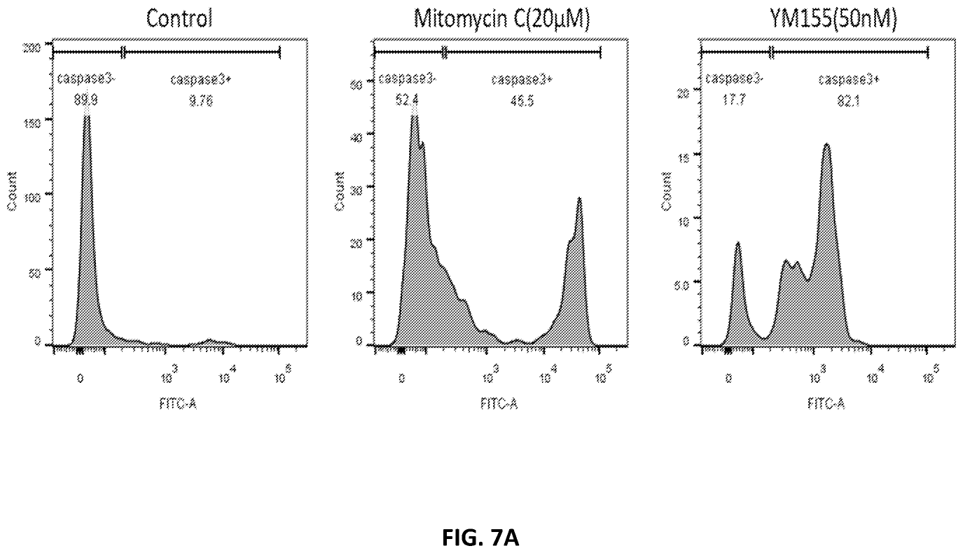

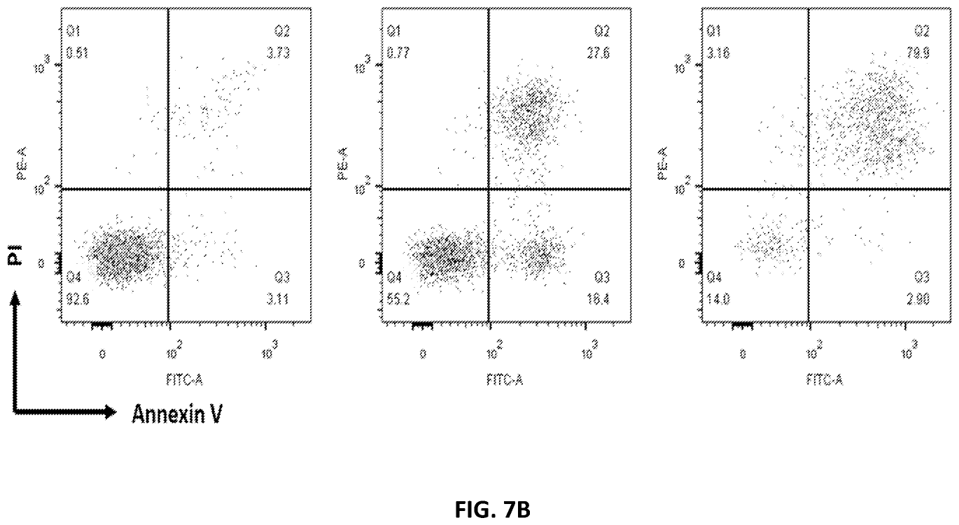

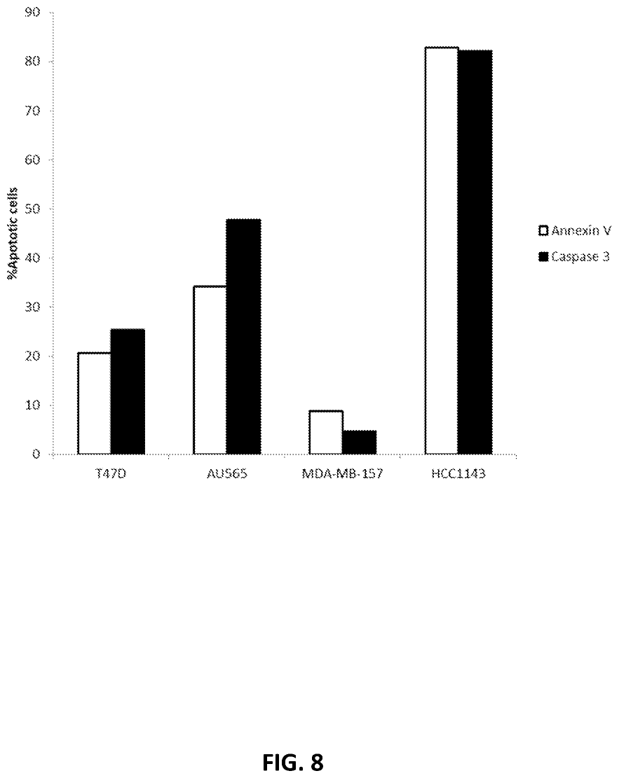

FIGS. 7A-7B show exemplary results of an apoptosis assay with HCC1143 cells using mitomycin C as a positive control; 7A shows the Caspase 3 activity assay, and 7B shows the Annexin V apoptosis assay. FIG. 8 shows the analysis of apoptosis in YM155-treated breast cancer cells by Annexin V and Caspase 3 assays. Table E2 shows the IC₅₀ values for growth inhibition and apoptosis induced by YM155 treatment in breast cancer cell lines, correlated to NOTCH3 copy number for each cell line.

TABLE E2
NOTCH3 mutation status and YM155
IC₅₀ in human breast cancer cell lines

| | IC₅₀ | % Apoptosis | % Apoptosis | NOTCH3 Copy |
|---|---|---|---|---|
| Cell Line | (nM) | (Annexin V) | (Caspase 3) | Number |
| HCC1143 | 20.53 | 82.8 | 82.1 | 2.5475 |
| MDA-MB-157 | 91.63 | 8.8 | 4.74 | 0.0943 |
| AU565 | 52.62 | 34.26 | 47.7 | −0.2252 |
| T47D | 19.06 | 20.64 | 25.4 | −0.4696 |

As summarized in Table E2, YM155 inhibited the proliferation of breast cell lines with IC₅₀ values ranging from about 20 nM to about 90 nM. In HCC1143 cells, the majority of the cells (>82%) went into apoptosis (see FIGS. 7A-7B). In contrast, only a small fraction of the cells were apoptotic in MDA-MB-157, AU565, and T47D cells (see Table E2 and FIG. 8). The endogenous level of NOTCH ICNs appeared to correlate with sensitivity to YM155 for breast cancer cells. For instance, HCC1143 cells, which are NOTCH3 gene amplified (Table E2), had the highest expression level of the ICNs (see FIG. 6), and were also the most sensitive to YM155 induced growth arrest and apoptosis.

1. A method for treating a NOTCH-associated cancer in a subject in need thereof, comprising
administering YM155 monobromide [1-(2-Methoxyethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof,
thereby treating the NOTCH-associated cancer in the subject in need thereof.

2. The method of claim 1, comprising,
(a) determining (i) intracellular NOTCH (ICN) levels, (ii) NOTCH mutation status, and/or (iii) NOTCH gene copy number, in a sample of cancer tissue from the subject; and
(b) administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof, to the subject if (i) ICN levels in the cancer tissue are increased relative to a control or reference, (ii) if the cancer tissue comprises an activating NOTCH mutation relative to wild-type NOTCH, or (iii) if the NOTCH gene copy number is increased relative to that of a NOTCH copy gene reference.

3. The method of claim 1 or 2, comprising administering to the subject a chemotherapeutic agent excluding (or other than) YM155 monobromide if (i) ICN levels in the cancer tissue are not substantially increased or undetectable relative to the control or reference, (ii) if the cancer tissue lacks an activating NOTCH mutation relative to wild-type NOTCH, and/or (iii) if NOTCH gene copy number is not substantially increased relative to that of the NOTCH copy gene reference.

4. A method for predicting therapeutic response to YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof, in a subject with cancer, comprising
(a) determining (i) intracellular NOTCH (ICN) levels, (ii) NOTCH mutation status, and/or (iii) NOTCH gene copy number, in a sample of cancer tissue from the subject; and
(b) (i) characterizing the subject as responsive to YM155 monobromide therapy if (i) ICN levels in the cancer tissue are increased relative to a control or reference, (ii) if the cancer tissue comprises an activating NOTCH mutation relative to wild-type NOTCH, or (iii) if the NOTCH gene copy number is increased relative to that of a NOTCH copy gene reference; or
(ii) characterizing the subject as non-responsive to YM155 monobromide therapy if (i) ICN levels in the cancer tissue are not substantially increased or undetectable relative to the control or reference, (ii) if the cancer tissue lacks an activating NOTCH mutation relative to wild-type NOTCH, and/or (iii) if NOTCH gene copy number is not substantially increased relative to that of the NOTCH copy gene reference,
thereby predicting therapeutic response to YM155 monobromide in the subject with cancer.

5. The method of claim 3, comprising administering YM155 monobromide to the subject if the subject is characterized as responsive to YM155 monobromide therapy.

6. The method of claim 3, comprising administering to the subject a chemotherapeutic agent excluding YM155 monobromide if the subject is characterized as non-responsive to YM155 monobromide therapy.

7. The method of any one of claims 1-6, wherein the NOTCH is NOTCH1, and wherein the ICN levels are intracellular NOTCH1 (ICN1) levels.

8. The method of any one of claims 1-7, wherein the NOTCH is NOTCH3, and wherein the ICN levels are intracellular NOTCH3 (ICN3) levels.

9. The method of any one of claims 1-8, comprising determining ICN levels, optionally ICN1 or ICN3 levels, in sample of cancer tissue by immunohistochemistry (IHC) optionally chromogenic or fluorescent IHC, enzyme linked immunosorbent assay (ELISA), or Western blot on a human NOTCH protein or gene.

10. The method of any one of claims 1-9, comprising administering YM155 to the subject if ICN levels, optionally ICN1 or ICN3 levels, in the cancer tissue are increased by about or at least about 1.2, 1.3, 1.4, 1.5, 2, 3, 4, 5, 6, 7, 8, 9, or 10, 50, or 100-fold or more relative to the ICN levels of the control or reference, optionally wherein the control is a healthy tissue.

11. The method of any one of claims 1-10, comprising determining NOTCH mutation status, optionally NOTCH1 or NOTCH3 mutation status, in the sample of cancer tissue in situ hybridization (ISH), fluorescence in situ hybridization (FISH), whole exome sequencing (WES), single nucleotide polymorphism (SNP) array, next generation sequencing (NGS), or comparative genome hybridization (CGH) on a human NOTCH protein or gene.

12. The method of any one of claims 1-11, wherein the NOTCH is NOTCH1, and wherein the activating NOTCH1 mutation is a ligand-independent activation (LIA) NOTCH1 mutant or an impaired degradation (ID) of NOTCH1 mutant.

13. The method of claim 12, wherein the activating NOTCH1 mutation is selected from one or more of one or more of a heterodimerization domain (HD) mutant, optionally a single nucleotide polymorphism (SNP) in the HD or a small in-frame insertion or deletion in the HD, a juxtamembrane expansion (JME) allele, and a C-terminal Pro-Glu-Ser-Thr-rich (PEST) domain mutant.

14. The method of any one of claims 1-13, wherein the NOTCH is NOTCH3, and wherein the activating NOTCH3 mutation is a C-terminal Pro-Glu-Ser-Thr-rich (PEST) domain mutant.

15. The method of any one of claims 1-14, comprising determining NOTCH gene copy number, optionally NOTCH1 or NOTCH3 gene copy number, in the sample of cancer tissue by array comparative genome hybridization (aCGH), single nucleotide polymorphism (SNP) array, copy number variation (CNV) sequencing, or multiplex ligation-dependent probe amplification (MLPA).

16. The method of any one of claims 1-15, wherein the NOTCH gene copy number, optionally NOTCH1 or NOTCH3 gene copy number, in the sample of cancer tissue is increased by about or at least about 1.5, 2, 3, 4, 5, 6, 7, 8, 9, or 10-fold relative to that of the NOTCH gene copy number reference.

17. The method of any one of claims 1-16, comprising obtaining the NOTCH gene copy number reference, optionally NOTCH1 or NOTCH3 gene copy number reference, from a database, or determining the NOTCH gene copy number reference from a non-cancerous tissue from a control, optionally by aCGH, SNP array, CNV sequence, or MLPA.

18. The method of any one of claims 1-17, comprising obtaining the sample of cancer tissue from the subject.

19. The method of any one of claims 1-18, wherein the sample of cancer tissue is a blood sample, a surgical sample, a biopsy sample, a pleural effusion sample, or an ascetic fluid sample obtained from the subject, optionally selected from one or more of a white blood cell, breast, lung, gastrointestinal (stomach, colon, rectal), ovarian, pancreatic, liver, bladder, cervical, neuronal, uterine, salivary gland, kidney, prostate, thyroid, or muscle tissue sample.

20. The method of any one of claims 1-19, wherein the subject is a human subject.

21. The method of any one of claims 1-20, wherein the cancer is selected from one or more of a leukemia, lymphoma (including Hodgkin's lymphoma and Non-Hodgkin's lymphoma), carcinoma, sarcoma such as rhabdomyosarcoma for example, alveolar rhabdomyosarcoma, (including sarcoma originating in the bones, tendons, cartilage, muscle, fat, fibrous, blood vessels, adipose, and/or connective tissue), breast cancer (including metastatic breast cancer), lung cancer (including non-small cell lung cancer, small cell lung cancer, adenocarcinoma, and squamous carcinoma of the lung), neuroblastoma, medulloblastoma, astrocytoma, glioblastoma multiforme, retinoblastoma, myeloma, adenosquamous carcinoma, carcinosarcoma, mixed mesodermal tumor, teratocarcinoma), gastrointestinal cancer, stomach cancer, colorectal cancer, colon cancer, rectal cancer, ovarian cancer, pancreatic cancer, liver cancer, bladder cancer, cervical cancer, glioblastoma, uterine carcinoma, salivary gland carcinoma, kidney or renal cancer (e.g., Wilm's tumor), prostate cancer, thyroid cancer, and head and neck cancer.

22. The method of claim 21, wherein the cancer is a leukemia or lymphoma, optionally selected from T-cell lymphoblastic leukemia (T-ALL), T-cell lymphoblastic lymphoma (T-LBL), and chronic lymphocytic leukemia (CLL), a non small cell lung cancer, or a breast cancer.

23. The method of claim 22, wherein the T-ALL is a NOTCH1-associated T-ALL, or a NOTCH3-associated T-ALL.

24. The method of claim 22, wherein the breast cancer is NOTCH-1 associated breast cancer, or a NOTCH3-associated breast cancer, optionally triple-negative breast cancer.

25. Use of a diagnostic kit for determining therapeutic response to YM155 monobromide [1-(2-Methoxy ethyl)-2-methyl-4,9-dioxo-3-(pyrazin-2-ylmethyl)-4,9-dihydro-1H-naphtho [2,3-d] imidazolium bromide], or an analog, derivative, or pharmaceutically acceptable salt thereof, therapy in a subject with cancer, comprising means for determining (i) intracellular NOTCH (ICN) levels, (ii) NOTCH mutation status, and/or (iii) NOTCH gene copy number, in a sample of cancer tissue from the subject, including cancer tissue and non-cancerous tissue.

26. The use of claim 25, wherein the NOTCH is NOTCH1.

27. The use of claim 25 or 26, wherein the NOTCH is NOTCH3.

28. The use of any one of claims 25-27, wherein the means for determining ICN levels comprise reagents for performing a diagnostic assay selected from one or more of immunohistochemistry (IHC) optionally chromogenic or fluorescent IHC, enzyme linked immunosorbent assay (ELISA), or Western blot on a human NOTCH protein.

29. The use of any one of claims 25-28, wherein the means for determining NOTCH mutation status comprise reagents for performing a diagnostic assay selected from one or more of in situ hybridization (ISH), fluorescence in situ hybridization (FISH), whole exome sequencing (WES), single nucleotide polymorphism (SNP) array, next generation sequencing (NGS), or comparative genome hybridization (CGH) on a human NOTCH protein or gene.

30. The use of any one of claims 25-29, wherein the means for determining NOTCH gene copy number comprise reagents for performing a diagnostic assay selected from one or more of array comparative genome hybridization (aCGH), single nucleotide polymorphism (SNP) array, copy number variation (CNV) sequencing, or multiplex ligation-dependent probe amplification (MLPA).

31. The use of any one of claims 25-30, wherein the cancer is selected from one or more of a leukemia, lymphoma (including Hodgkin's lymphoma and Non-Hodgkin's lymphoma), carcinoma, sarcoma such as rhabdomyosarcoma for example, alveolar rhabdomyosarcoma, (including sarcoma originating in the bones, tendons, cartilage, muscle, fat, fibrous, blood vessels, adipose, and/or connective tissue), breast cancer (including metastatic breast cancer), lung cancer (including non-small cell lung cancer, small cell lung cancer, adenocarcinoma, and squamous carcinoma of the lung), neuroblastoma, medulloblastoma, astrocytoma, glioblastoma multiforme, retinoblastoma, myeloma, adenosquamous carcinoma, carcinosarcoma, mixed mesodermal tumor, teratocarcinoma), gastrointestinal cancer, stomach cancer, colorectal cancer, colon cancer, rectal cancer, ovarian cancer, pancreatic cancer, liver cancer, bladder cancer, cervical cancer, glioblastoma, uterine carcinoma, salivary gland carcinoma, kidney or renal cancer (e.g., Wilm's tumor), prostate cancer, thyroid cancer, and head and neck cancer.

32. The use of claim 31, wherein the cancer is a leukemia or lymphoma, optionally selected from T-cell lymphoblastic leukemia (T-ALL), T-cell lymphoblastic lymphoma (T-LBL), chronic lymphocytic leukemia (CLL), a non small cell lung cancer, or a breast cancer.

33. The use of claim 32, wherein the T-ALL is a NOTCH1-associated T-ALL, or a NOTCH3-associated T-ALL.

34. The use of claim 32, wherein the breast cancer is NOTCH-1 associated breast cancer, or a NOTCH3-associated breast cancer, optionally triple-negative breast cancer.

35. A patient care kit, comprising:
(a) means for determining (i) intracellular NOTCH (ICN) levels, (ii) NOTCH mutation status, and/or (iii) NOTCH gene copy number, in a sample of tissue from a subject, including cancer tissue and non-cancerous tissue; and
(b) YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof.

36. The patient care kit of claim 35, wherein the NOTCH is NOTCH1.

37. The patient care kit of claim 35 or 36, wherein the NOTCH is NOTCH3.

38. The patient care kit of any one of claims 35-37, wherein the means for determining ICN levels comprise reagents for performing a diagnostic assay selected from one or more of immunohistochemistry (IHC) optionally chromogenic or fluorescent IHC, enzyme linked immunosorbent assay (ELISA), or Western blot on a human NOTCH protein.

39. The patient care kit of any one of claims 35-38, wherein the means for determining NOTCH mutation status comprise reagents for performing a diagnostic assay selected from one or more of in situ hybridization (ISH), fluorescence in situ hybridization (FISH), whole exome sequencing (WES), single nucleotide polymorphism (SNP) array, next generation sequencing (NGS), or comparative genome hybridization (CGH) on a human NOTCH protein or gene.

40. The patient care kit of any one of claims 35-39, wherein the means for determining NOTCH gene copy number comprise reagents for performing a diagnostic assay selected from one or more of array comparative genome hybridization (aCGH), single nucleotide polymorphism (SNP) array, copy number variation (CNV) sequencing, or multiplex ligation-dependent probe amplification (MLPA).

41. A pharmaceutical composition for use in treating a NOTCH-associated cancer, comprising YM155 monobromide [1-(2-Methoxy ethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof.

42. The pharmaceutical composition for use according to claim 41, wherein the NOTCH-associated cancer is a NOTCH1-associated cancer, and/or a NOTCH3-associated cancer, optionally a T-ALL or a breast cancer.

43. Use of a composition in the preparation of a medicament for treating a NOTCH-associated cancer, comprising YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog, derivative, or pharmaceutically acceptable salt thereof.

44. The use according to claim 43, wherein the NOTCH-associated cancer is a NOTCH1-associated cancer, and/or a NOTCH3-associated cancer, optionally a T-ALL or a breast cancer.
